(19) United States
(12) Patent Application Publication
(10) Pub. No.: US 2026/0321015 A1
(43) Pub. Date: Oct. 15, 2026

(54) AI-BASED MULTIMODAL COMPUTER VISION SYSTEM FOR ANALYSIS, SUMMARIZATION, AND MONITORING OF SURGICAL AND NON-SURGICAL MEDICAL PROCEDURES

(71) Applicant: Clinical Video, Inc. DBA VisionMed, Far Hills, NJ (US)

(72) Inventor: Hamza Hasan, Far Hills, NJ (US)

(21) Appl. No.: 19/639,414

(22) Filed: Apr. 6, 2026

Related U.S. Application Data
(60) Provisional application No. 63/785,694, filed on Apr. 9, 2025.

(51) Int. Cl. G06V 20/52; A61B 34/32; G06F 3/01; G06F 3/16; G06V 10/26; G06V 10/764; G06V 10/774; G06V 10/98; G16H 20/30

(52) U.S. Cl. CPC G06V 20/52; A61B 34/32; G06F 3/015; G06F 3/167; G06V 10/26; G06V 10/764; G06V 10/774; G06V 10/993; G16H 20/30; G06V 2201/03

(57) ABSTRACT

Systems and methods for processing and using video. The methods comprise: obtaining, by a processor, a maximum number of input tokens that are allowed to be ingested by an AI analysis engine per session for a video quality associated with the video; determining a maximum desired temporal duration for a video segment based on the maximum number of input tokens and data analytics for other data to be input into the AI analysis engine along with video; determining a total number of video segments that the video can be partitioned into based on the video temporal duration and the maximum desired temporal duration; determining possible temporal durations for the video segments; and segmenting the video into a plurality of video segments based on the total number of video segments, the possible temporal durations for the video segments, and/or task types associated with frames of the video.

### CROSS-REFERENCE TO RELATED APPLICATION

The present application claims priority to and the benefit of U.S. Provisional Application No. 63/785,694 which was filed on Apr. 9, 2025. The content of this Provisional Application is incorporated in its entirety herein by reference.

### BACKGROUND

### Description of the Related Art

Existing technologies for documenting and analyzing medical procedures are limited in their ability to support multimodal data types and often lack real-time monitoring and compliance-checking capabilities. Current systems typically capture only video or basic audio, leaving out other critical data types such as patient vitals, historical context, and detailed imaging. These systems fail to integrate this data effectively, limiting their ability to provide actionable insights and safety alerts in real-time. Moreover, traditional video documentation systems do not offer detailed analytics, compliance checking, or anomaly detection capabilities, which are increasingly needed to improve patient outcomes, clinician efficiency, and procedural safety.

### SUMMARY

The present document concerns implementing systems and methods for processing and using video of a medical procedure in progress. The methods comprise: obtaining, by a processor, a maximum number of input tokens that are allowed to be ingested by an artificial intelligence (AI) analysis engine per session for a video quality associated with the video; determining, by the processor, a maximum desired temporal duration for a video segment based on the maximum number of input tokens and data analytics for other data to be input into the AI analysis engine along with video; determining, by the processor, a total number of video segments that the video can be partitioned into based on the video temporal duration and the maximum desired temporal duration; determining, by the processor, possible temporal durations for the video segments; segmenting, by the processor, the video into a plurality of video segments based on the total number of video segments, the possible temporal durations for the video segments, and/or task types associated with frames of the video; analyzing, by the AI analysis engine, a video segment of the plurality of video segments to monitor compliance of the medical procedure, automate documentation of the medical procedure, and/or detect or predict a safety issue during the medical procedure; and continuing the medical procedure by controlling autonomous operations of an avatar or a robotic system based on at least an output of the AI analysis engine.

The present document also concerns a system comprising: an avatar or a robotic system; an AI analysis engine; a processor configured to process and use video of a medical procedure in progress to control autonomous operations of the avatar or the robotic system; and a non-transitory computer-readable medium that stores instructions. The non-transitory computer-readable medium is configured to, when executed by the processor, cause the processor to perform operations comprising: obtaining a maximum number of input tokens that are allowed to be ingested by the AI analysis engine per session for a video quality associated with the video; determining a maximum desired temporal duration for a video segment based on the maximum number of input tokens and data analytics for other data to be input into an AI analysis engine along with video; determining a total number of video segments that the video can be partitioned into based on the video temporal duration and the maximum desired temporal duration; determining possible temporal durations for the video segments; and segmenting the video into a plurality of video segments based on the total number of video segments, the possible temporal durations for the video segments, and/or task types associated with frames of the video. The AI analysis engine analyzes a video segment of the plurality of video segments to monitor compliance of the medical procedure, automate documentation of the medical procedure, and/or detect or predict a safety issue during the medical procedure. The medical procedure is continued by controlling autonomous operations of the avatar or the robotic system based on at least an output of the AI analysis engine.

### BRIEF DESCRIPTION OF THE DRAWINGS

The present solution will be described with reference to the following drawing figures, in which like numerals represent like items throughout the figures.

FIG. 1 provides a block diagram of an illustrative system implementing the present solution.

FIG. 2 provides a block diagram of illustrative modules that may be implemented by the system of FIG. 1.

FIG. 3 provides a flow diagram of an illustrative method for multimodal analysis.

FIG. 4 provides a flow diagram of an illustrative method for segmenting video.

FIGS. 5A-5B (collectively referred to as “FIG. 5”) provides a flow diagram of another illustrative method for segmenting video.

FIG. 6 provides a flow diagram of another illustration method for operating a system in accordance with the present solution.

FIG. 7 provides an illustration showing a cascaded reasoning train architecture for an artificial intelligence (AI) engine or analysis engine of the present solution.

FIGS. 8A-8C (collectively referred to as “FIG. 8”) provides illustrations showing different timing schedules for reasoning trains of the AI engine or analysis engine.

FIG. 9 provides a flow diagram of an illustrative method for operating a medical system in accordance with the present solution.

FIG. 10 provides a flow diagram of an illustrative method for processing and using video of a medical procedure in progress.

FIG. 11 provides a block diagram of a computing device.

### DETAILED DESCRIPTION

The present document concerns an AI based multimodal computer vision system designed for analyzing surgical and non-surgical medical procedures through video files, live video feeds, medical imaging, audio, and other medical data such as patient vitals and historical data. The system comprises multiple components for ingesting and processing data across modalities, including video stabilization, noise reduction, image segmentation, audio transcription, and real-time synchronization of each modality. An AI analysis engine then assesses procedural phases, detects anomalies, monitors adherence to clinical protocols, and outputs a comprehensive summary and analytics of the procedure. Outputs are provided in various document formats, such as Excel, Word, PDF, JSON, and are compatible with electronic health record (EHR) systems. The present solution supports real-time monitoring for active procedural analysis, allowing the clinical team to receive real-time alerts, safety flags, and compliance insights, ultimately improving procedural safety, accuracy, and efficiency.

Conventional methods of documenting and monitoring medical procedures face several limitations. With regard to a data limitation, systems tend to focus on one modality (e.g., video only) rather than integrating multiple forms of data, missing critical insights derived from the combined analysis. Conventional solutions also have real-time processing shortcomings. Many systems lack real-time monitoring capabilities, preventing them from issuing timely alerts and safety recommendations during procedures. In terms of compliance monitoring, the absence of protocol adherence checks limits the value of the documentation, as it fails to address potential risks associated with non-standard procedural steps. Conventional solutions require labor-intensive documentation. Manual documentation is time-consuming and prone to error, impacting procedural accuracy and clinician workload. The present solution seeks to address these limitations by providing a multimodal AI system capable of processing multiple data inputs, monitoring compliance, issuing real-time safety alerts, and automating documentation.

Objectives of the present solution are: (1) to provide a multimodal AI system that ingests video, medical imaging, audio, patient vitals, and historical data, integrating them into a comprehensive procedural analysis; (2) to enable real-time monitoring and safety flagging for active procedures, enhancing compliance and safety; and (3) to automate the generation of procedural summaries, analytics, and compliance insights in multiple formats for EHR integration.

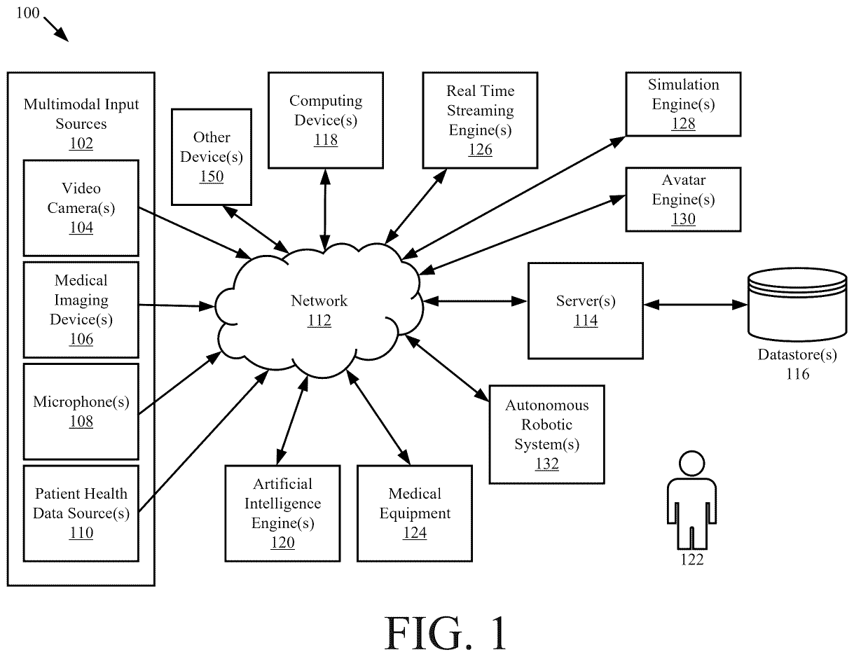

FIG. 1 provides a block diagram of an illustrative system 100 implementing the present solution. System 100 comprises an AI-driven system that processes and analyzes multiple forms of medical data (e.g., video files, imaging data, audio inputs, patient vitals, historical data from surgical and non-surgical medical procedures, and historical records). The AI-driven system is configured to provide real-time and post-procedure documentation, safety monitoring, and compliance insights.

System 100 can be used in various applications. The applications include, but are not limited to, surgical procedure applications, outpatient procedure applications, and/or post-operative analysis applications. In surgical procedure applications, real-time video and audio monitoring may be performed with vitals integration, showing how safety alerts are issued. In outpatient procedure applications, the system can demonstrate how multimodal analysis produces a comprehensive summary with compliance metrics. In post-operative analysis applications, the system may analyze recorded video for trend analysis and protocol adherence.

System 100 implements a transformative approach to medical procedure analysis, integrating multimodal data to enhance safety, compliance, and documentation in real-time. System 100 has many advantages. Such advantages include, but are not limited to, comprehensive analysis (e.g., integrates video, imaging, audio, and patient data, offering a holistic procedural view), real-time monitoring (e.g., to provide real-time alerts to enhance safety), efficient documentation (e.g., via automation of procedural documentation to reduce clinician workload), and enhanced decision-making (e.g., via real-time multimodal analysis supports clinical teams in active monitoring).

System 100 comprises multimodal input sources 102 communicatively coupled to server(s) 114 via network 112. The multimodal input sources 102 can be located in doctor office(s), hospital(s), clinic(s), therapist office(s), urgent care facility(ies), operating room(s), delivery room(s), and/or any other patient related building or room. The multimodal input sources 102 can include, but are not limited to, video camera(s) 104, medical imaging device(s) 106, microphone(s) 108, patient health data source(s) 110, and/or other data source(s) (e.g., wearable health monitoring device(s), virtual reality glasses, etc.). The video camera(s) 104 may be mounted to a wall or other structure, included in virtual reality glasses, mounted on a medical equipment, and/or mounted on medical instruction(s) (e.g., an end of an endoscope). Patient health data source(s) 110 may include, but are not limited to, hospital systems, doctor office systems, therapist office systems, insurance company systems, clinic systems, urgent care service provider systems, and/or other health and medical service provider systems. The patient health data can include, but is not limited to, patient name, birth date, location, age, diagnosis, previous medical treatments, scheduled medical procedure(s), medication(s), doctor(s), family medical history, and any other patient health related information. Network 112 can include, but is not limited to, the Internet, an Intranet, and/or any other known or to be known network. The server(s) 114 is(are) configured to facilitate storage of data received from multimodal input sources 102 in datastore(s) 116. Datastore(s) 116 may be accessible to computing device(s) 118.

Computing device(s) 118 is(are) generally configured to provide a novel interface between the multimodal input sources 102 and AI engine(s) 120. In this regard, computing device(s) 118 is(are) configured to access, retrieve and pre-process data stored in datastore 116 to put in a format suitable for processing by AI engine(s) 120 that are trained to at least generate electronic medical procedure note documents. The pre-processing operations can include, but are not limited to: performing video segmentation; performing image and/or video analysis to detect and/or flag medical instruments, medical equipment, body parts, nurses, doctors, patient(s) represented in image(s) and/or video(s); automated audio transcription; detecting occurrences of events based on different types of data (e.g., audio data, textual data (e.g., treatment plan or patient record text), imaging data, video(s), etc.); and/or temporal correlation of different types of data (e.g., by adding timestamps to link segments of video to the detected events or tasks, adding timestamps to the video and/or other data for linking segments of video to types of tasks or events specified or indicated by the other data).

The AI engine(s) 120 may comprise neural networks and/or other deep learning algorithms. For example, the AI engine(s) 120 may comprise(s) an open AI system such as a large language model(s) (LLM(s)) that can generate contextual human language based on content of video(s). The LLMs are trained to recognize anatomy, medical instruments, and medical procedures within video(s). Contextual information may be provided to the LLMs to improve the efficiency of the LLMs operations. For example, the contextual information can include the theme of video (e.g., general medical procedure), medical procedure type (e.g., ACL repair or endoscopy), and/or body part at issue (e.g., knee).

Operations of AI engine(s) 120 may be better understood through an illustrative scenario in which a patient 122 has an anterior cruciate ligament (ACL) repair. Computing device 118 pre-processes and inputs the following data into the AI engine(s) 120—an image of the patient's MRI, the patient's chart including a pre-operative clinical assessment, and video(s) of the actual medical procedure to repair the ACL. Given the context of the ACL repair seen in the MRI plus the pre-operative clinical assessment and plus the actual procedure that occurred, the AI engine(s) 120 prepares an electronic document comprising robust medical procedure notes with the understanding of what the tear is and what the pre-operative assessment of the patient was. The medical procedure notes may include a step-by-step of events that happened during the actual medical procedure shown in the video. The step-by-step of events may be presented in a list format. For example, the list of steps can include: (1) incision is made, (2) the wound is debrided or cleaned of dead or damaged tissue, (3) instrument inserted, (4) another incision made, (5) bone drilled, (7) insert medical device, and (8) close wound. The list of steps may be generated by the AI engine(s) 120 solely by reviewing the video(s) and adding other relevant information from the other non-video data (e.g., the patient's MRI and/or chart). A single video may be analyzed to generate the medical procedure notes. Alternatively, multiple videos may be analyzed to generate the medical procedure notes. The multiple videos can include a first video captured by a camera of the doctor's virtual reality glasses, and/or a second video captured by a camera located at a distal end of an endoscope. A first list of steps is generated by the AI engine(s) 120 based on the first video. A second list of steps is generated by the AI engine(s) 120 based on the second video. AI engine(s) 120 then combine(s) the first and second lists to produce the medical procedure notes comprising a single list or step-by-step of events. By adding the relevant information from other non-video data, the medical procedure notes are more robust than conventional medical procedure notes since they includes factors and/or background information as to why the patient had the medical procedure performed. The relevant information added to the medical procedure notes can include, but is not limited to, health status of the patient. The present solution is not limited to the particulars of this example.

The AI engine(s) 120 may additionally be trained to generate medical recommendations, a recover timeline, recommended timing for patient follow-up, recommended medications, recommended next steps for the doctor and/or patient, and/or a treatment plan with information for controlling parameters of medical equipment. The listed information may be added to the medical procedure notes.

Computing device 118, AI engine(s) 120 and/or real-time streaming engine(s) 126 may implement distinct modules that are each designed to preprocess and analyze specific data types. The core AI analysis engine then synchronizes and interprets these data points to generate a comprehensive overview of a given medical procedure.

Computing device 118 and/or avatar engine(s) 130 may implement hardware and/or software configured to facilitate integration of a live streaming avatar with system 100. Characteristics of the avatar may be customizable. The characteristics can include, but are not limited to, appearance, voice, gestures (or movements), and/or language. Also, the avatar may be configured based on, swapped in/out, or replaced with an avatar for another external software application (e.g., a video gaming avatar). The system may have a multilingual capability. The avatar may comprise a 2D icon or a 3D model of a person. The avatar may interact, communicate and express itself in a computing environment within the physics that are simulated. The avatar has vision capability outside of the surrounding environment through use of video captured by a camera that is indirectly communicatively coupled to the avatar engine(s) 130. The camera may include, but is not limited to, a camera of a smart phone. The avatar may also have vision capability inside the surrounding environment and real-time audio streaming capability to verbally communicate with the user. The avatar may also be integrated into other applications and configured to facilitate transactions (e.g., payment transactions).

The avatar may assist users with inquiries and provide information to the users in response to their inquiries. In this regard, real-time video analysis may be streamed to the avatar. The avatar may be configured to read or otherwise auditorily present (e.g., via speech) and/or visually present (e.g., via gestures) information output from the AI engine(s) 120 and/or real-time streaming engine(s) 126. In some scenarios, the system compresses the entire real-time video stream into a searchable context window from which the avatar may retrieve relevant segments to answer user inquiries and/or questions about the medical procedure. The avatar may respond to commands from a user of system 100. The commands may include, but are not limited to, voice commands, gesture commands, or commands input via user input devices (e.g., keyboard, buttons, mouse, widgets, etc.).

Computing device(s) 118 can include, but is(are) not limited to, personal computers, tablets, laptop computers, desktop computers, personal digital assistants, and/or wearable computing devices. The wearable computing devices can include, but are not limited to, a headband, a headset, smart glasses, smart watches, and/or other wearable items. The avatar, analytics, real-time streaming video, and/or other information may be projected onto a surface of a heads-up display or other display of computing device(s) 118. The real-time streaming video may be output from the real-time streaming engine(s) 126. The analytics and/other information may be output from the AI engine(s) 120 and/or LLMs.

The surface of a heads-up display may be transparent so that a real-world environment is visible to the wearer of the wearable computing device. In some scenarios, the real-time streaming video is presented across all or significant portion of the transparent surface of the heads-up display. The avatar, analytics and/or other information may be overlaid on the real-time streaming video. In other scenarios, a real-world environment may be visible through the transparent surface of the heads-up display. In this way, a user may watch an event (e.g., a medical procedure), while having a direct line of sight of the avatar and/or analytics without having to look away from the real-world environment.

The relative arrangement of the real-time streaming video, avatar, analytics and/or other information on the display (e.g., transparent surface of heads-up display) may be customizable and/or variable based on certain criteria. A window showing the real-time streaming video, avatar, analytics and/or other information may be dragged across the display using a widget to allow for different arrangements or permutations thereof in or on the display. The relative arrangement of the avatar and analytics may be different during a medical procedure than during another type of event. During the medical procedure, the avatar may be presented on the right of the transparent surface, while the analytics are presented on the left side of the transparent surface. In contrast, the avatar may be presented on the left side, the bottom, or in the middle of the transparent surface during another type of event. The present solution is not limited to the particulars of these scenarios.

In some scenarios, the medical equipment may be a robot controlled by a remote operator or may be autonomously controlled by a controller. For example, a resident physician is performing surgery and an attending physician is watching the procedure from a remote location. The resident physician is wearing a headset on which an avatar and analytics are displayed so as to be overlaid on a transparent display through which a real-world environment that is visible to the resident physician. The attending physician may use a headset to view the avatar, analytics and/or real-time streaming video of the environment at the same time they are being output from the headset worn by the resident physician during the surgery. This allows the attending physician to view the procedure as it is ongoing, review the analytics and/or interact with the avatar from the remote location. Results from the attending physician's interaction with the avatar may be presented on the headset of the attending physician and/or the headset of the resident physician. The present solution is not limited to the particulars of this example.

The user may ask the avatar for recommendations. The questions and/or inquiries may include speech inputs that are captured by a microphone of the computing device(s) 118. For example, the resident physician may ask the avatar “Is this the right method that I am using to perform the procedure?”, “What is the next step of the procedure?”, and/or “What is the risk if I perform a particular step or action?”. In response to the question, the avatar engine(s) 130 may access and/or search a repository of video clips in datastore(s) 116 that contain relevant content. The video clips may then be displayed on the display to the user along with the avatar. The video clips may be overlayed through the headset without interrupting a procedure. The present solution is not limited to the particulars of this example.

The headset of computing device(s) 118 may include commercial off the shelf (COTS) headsets and/or specially designed headsets. The headset may include, but is not limited to, a headband, glasses, or goggles. The headset(s) may comprise a display, a camera, a microphone, a bone conduction speaker, brain-computer interface(s), a headlamp, a magnifying glass (e.g., a loupe), and/or clips. The bone conduction speaker presses against a user's skull when the headset is being worn. The bone conduction speaker uses transducers to convert audio signals into vibrations. The vibrations are transmitted through the user's skull bone to the user's inner ear. In this way, the user hears audio through these vibrations. This enables the user to hear ambient sound without obstruction to the user's ear canal. The brain computer interface(s) may comprise an electroencephalogram (EEG) for measuring brain activity and a processor configured to receive EEG impulse(s) for controlling the avatar and/or other software features of system 100. The other software features can include, but are not limited to, start recording video, stop recording video, start recording audio, stop recording audio, start displaying streaming video, stop displaying streaming video, start displaying analytics, and/or stop displaying analytics.

The headset of computing device(s) 118 may be configured to facilitate generation of a training dataset for use in training an autonomous robotic system 132. The autonomous robotic system can include, but is not limited to, an autonomous robotic surgery system. For example, the headset may be used to track hand movements of a surgeon while performing a medical procedure. The system may then generate a dataset that correlates between the tracked hand movements that the headset observes and tracked movements of surgical tool(s). The surgical tool movements may be tracked using sensors coupled to medical equipment 124. For example, the surgical tool movement may be tracked via an endoscope feed, i.e., a real-time video stream from a camera attached to an endoscope. Such a correlated dataset may be generated for a plurality of medical procedures or surgeries. The correlated datasets may be used to train an autonomous surgical system 132. This allows robotic platforms to learn from annotated real-world surgical video streams. The present solution is not limited to the particulars of this example. The analytics and/or training datasets may additionally or alternatively be used to validate workflows, recommend workflow improvements (e.g., to hospitals, clinics, or other business entities), facilitate compliance monitoring and analytics, facilitate procedural benchmarking, and/or facilitate predictive insights.

Real-time streaming engine(s) 126 is configured to continuously process and analyze data streams to extract actionable insights in near-instantaneous time. The analysis may assess procedural phases, detect anomalies, monitor adherence to clinical protocols, and output a comprehensive summary and analytics of an event or procedure. Real-time streaming engine(s) 126 may be audio based, text based, video based, and/or multimodal. Real-time streaming engine(s) 126 outputs real-time streaming video that may be presented by the computing device(s) 118. The system may thread between the real-time streaming engine(s) 126 (e.g., streaming APIs) and the AI engine(s) 120 (e.g., LLM APIs). The real-time streaming engine(s) 126 (e.g., streaming APIs) may be continuously running, while the AI engine(s) 120 (e.g., LLM APIs) may be periodically run. The real-time streaming engine(s) 126 (e.g., streaming APIs) may send requests to the AI engine(s) 120 (e.g., LLM APIs) for analytics. The output of the AI engine(s) 120 (e.g., LLM APIs) may be input into the real-time streaming engine(s) 126 (e.g., streaming APIs) and output along with the real-time streaming video. For example, in a surgery context, the real-time streaming engine(s) 126 (e.g., streaming APIs) collect data (e.g., video, audio or other data), performs an algorithm, and causes the display of a live stream output on the computing device(s) 118. The AI engine(s) 120 (e.g., LLM APIs) may be running in the background. The output of the AI engine(s) 120 (e.g., LLM APIs) is provided as an input to the real-time streaming engine(s) 126 (e.g., streaming APIs). The real-time streaming engine(s) 126 (e.g., streaming APIs) uses this input to generate its output. In this case, the user may be presented, for example, a live streaming video, audio or other data with the LLM output, context or content overlaid on the live streaming video. The present solution is not limited to the particulars of this example.

Computing device(s) 118 and/or simulation engine(s) 128 may implement hardware and/or software configured to facilitate training physicians and residents using simulated patients, enabling immersive medical scenario simulations in general with real-time analytics and feedback. In this regard, the simulation engine(s) 128 may be configured to generate a simulated environment in which the physicians may interact with the simulated patients, have conversations about their health (e.g., have simulated end of life conversations), and/or perform simulated procedures. The simulation engine(s) 128 may record data as the physicians interact with the simulated patients and/or perform simulated procedures. The data may be input into the AI engine(s) 120 (e.g., LLM APIs). The outputs of the AI engine(s) 120 (e.g., LLM APIs) may be presented to the physicians in the simulated environment and/or overlayed on the simulated environment displayed on a heads-up display. The outputs of the AI engine(s) 120 (e.g., LLM APIs) may provide feedback on the physicians' communication skills. The feedback can include, but is not limited to, recommendations for improving communication with patients, and/or recommendations for improving actions and/or tasks in the simulated environment.

The present solution may be integrated with other software applications. For example, the present solution may be integrated with live collaboration software applications (such as Zoom, Google Meet, Teams, etc.). The present solution may provide an autonomous surgical platform capable of performing procedures independently with human oversight.

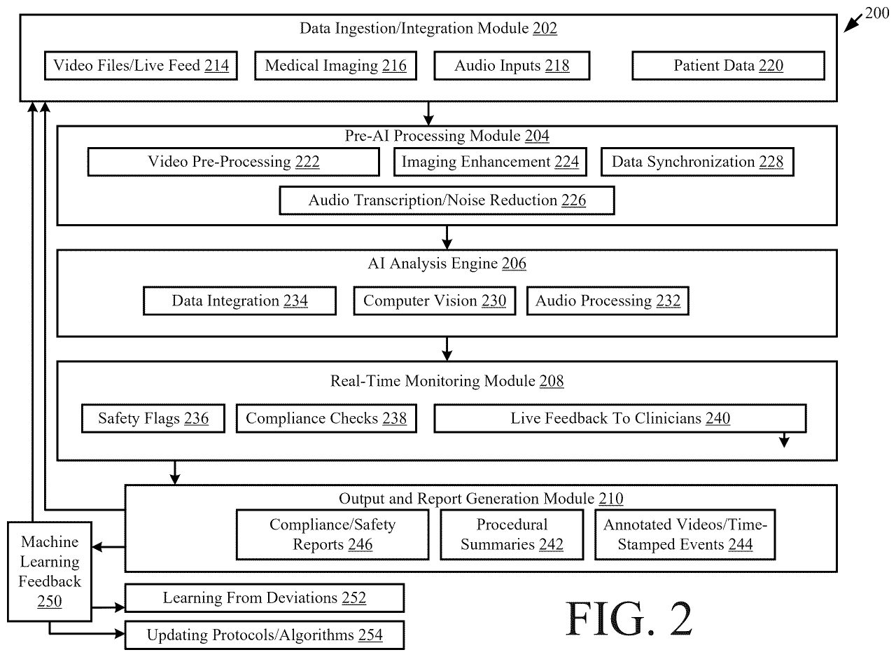

FIG. 2 provides an illustration showing modules 200 that may be implemented by computing device 118. The modules 200 comprise a data ingestion/integration module 202, a pre-AI processing module 204, an AI analysis engine 206, a real-time monitoring module 208, and an output and report generating module 210. Modules 200 may include more or less modules than shown.

During operation, data ingestion/integration module 202 receives multimodal inputs. The pre-AI processing module 204 pre-processes each data type of multimodal inputs, ensuring that noise, irrelevant data, and other artifacts are removed. AI analysis engine 206 then performs an in-depth analysis on each modality, detecting procedural phases, identifying key anatomical structures, and interpreting audio cues. All data is synchronized and compiled into a procedural summary, which includes compliance metrics, safety flags, and a breakdown of procedural steps with time-stamped actions.

Data ingestion/integration module 202 is configured to support various data types including video, medical imaging, audio and/or patient data. Data ingestion/integration module 202 is configured to receive and/or handle pre-recorded videos, live video feeds, medical imaging, audio, and patient health data. The pre-recorded videos or live video feeds may be received from procedural cameras. The medical imaging data can include, but is not limited to, digital imaging and communications (DICOM) data, X-ray images, magnetic resonance imaging (MRI) data, and computed tomography (CT) scan data. The audio may be captured by microphones and passed to the data ingestion/integration module 202 for processing in real-time or near-real time. The audio can include, but is not limited to, surgeon instructions and procedural sounds. The patient health data can include, but is not limited to, patient vital data (e.g., heart rate, blood pressure, etc.) captured by sensors, and/or historical medical records.

Data ingestion/integration module 202 comprises a video files and live feed submodule 214, a medical imaging submodule 216, an audio input submodule 218, and a patient data submodule 220. Each of the submodules 214-220 is configured to receive and process a respective type of input data.

Pre-AI processing module 204 is configured to preprocess data. Video preprocessing may involve stabilization, noise reduction, and segmentation ensure data quality for analysis. Imaging processing may involve resolution enhancement, feature extraction, and contrast adjustment for clearer imaging. Audio processing may involve speech-to-text transcription, noise reduction, and synchronization with video. Data synchronization may involve aligning temporal data from all modalities, creating a unified timeline.

Pre-AI processing module 204 comprises a video pre-processing submodule 222, an imaging enhancement submodule 224, an audio transcription and noise reduction submodule 226, and a data synchronization submodule 228. Submodule 222 is configured to receive a video, stabilize the video (e.g., by tracking a salient feature in image(s) and use this as an anchor point to cancel out all perturbations relative to it), and/or extract frames from the video file. Submodule 224 is configured to perform image enhancement for improving image visual quality by adjusting features like brightness, contrast, sharpness and/or color. Submodule 226 is configured to process audio of the video to reduce noise, and/or convert spoken words from the video recording into written text. Submodule 228 is configured to synchronize data across locations or systems by updating and maintaining the same information across all sources. This is performed to avoid discrepancies and ensure data integrity.

Pre-AI processing module 204 may optionally be configured to perform object detection and annotate the video by adding overlaid shapes around the detected objects (e.g., medical instruments, or polyps). Any known or to be known object detection algorithm can be used here.

Pre-AI processing module 204 may also optionally be configured to partition the video into segments. Each segment can include a set of consecutive frames. The size of each segment may be determined based on (1) video analytics (e.g., quality of video, frames per second, total number of frames in the video, and total duration of video), and (2) a known maximum number of input tokens that are allowed to be ingested by a given AI analysis engine per session. For example, a video has a total length of forty-five minutes and the given AI engine can process a maximum of ten minutes of video per session. Accordingly, the frames are segmented to produce a first segment comprising ten minutes of video, a second segment comprising ten minutes of video, a third segment comprising ten minutes of video, a fourth segment comprising ten minutes of video, and a fifth segment comprising five minutes of video. The present solution is not limited to the particulars of this example.

AI analysis engine 206 comprises a computer vision submodule 230 implementing AI that enables a computer to see and interpret images and videos, an audio processing submodule 232 configured to detect, manipulate and/or modify speech and contextual sounds, and a data integration submodule 234 for combining and/or synchronizing multimodal data from various sources. Vision submodule 230 may be configured to use deep learning models to detect procedural phases, anatomical regions, and instruments. For example, vision submodule 230 may implement machine learning model(s) configured to process segments of frames in serial sessions. A first segment of frames is processed during a session to produce a first sub-list of step-by-step events. A second segment of frames is processed during a next session to process a second sub-list of step-by-step events. The first and second sub-lists are combined to form a single consolidated list of step-by-step events. The consolidated list of step-by-step events is then used to generate electronic medical procedure notes for the entire medical procedure. The electronic medical procedure notes may also include time stamps for steps, instrument identifications, and/or billing codes. The present solution is not limited to the particulars of this example.

Audio processing submodule 232 may employ natural language processing (NLP) and contextual analysis to interpret verbal commands and other audio cues. Data integration submodule 234 may be configured to merge insights from video, imaging, audio, and patient data for cohesive analysis.

The real-time monitoring module 208 is configured to: monitor for adherence to medical protocols, issuing real-time alerts when anomalies are detected; and/or continuously assesses procedural compliance and issues safety alerts based on predefined protocols, such as the length of time instruments are used or patient vital sign changes that deviate from the norm. The real-time monitoring module 208 comprises a safety flags submodule 236, a compliance checks submodule 238, and a live feedback to clinicians submodule 240. Safety flags submodule 236 is configured to flag safety issues detected in medical procedures, treatment plans, data transfer systems or protocols, and/or the like. Compliance checks submodule 238 is configured to check compliance of medical procedures, treatment plans, data transfer systems or protocols, and/or other system operations. Submodule 240 is configured to feedback live video and/or other data to clinicians and/or other individuals.

The output and report generating module 210 is configured to generate output documents in multiple formats and/or for integration with EHRs, ensuring compatibility with clinical workflows. The formats can include, but are not limited to, Excel, Word, PDF, and/or JSON. Module 210 comprises a compliance/safety reports submodule 246, a procedural summaries submodule 242, and an annotated videos/time-stamped events submodule 244.

A machine learning feedback loop is provided so that the AI can learn from prior analyses, refining its algorithms to improve accuracy and efficiency over time. Annotations from clinicians may be integrated to enhance the system's understanding of procedural nuances. In this regard, outputs from one or more of these submodules 244-246 may be passed to machine learning feedback block 250 for use in further training machine learning algorithms and/or updating machine learning models. The machine learning algorithms and/or models may be configured to learn from deviations as shown by block 252. The feedback information may also be used to update protocols and/or other algorithms as shown by block 254.

The AI of module(s) 206, 208 may operate in a continuous mode for live video feeds, processing data at set intervals, and providing real-time feedback and safety alerts. Modules 200 may have a scalable architecture for AI analysis. Module 202 may be configured to dynamically segment input data to ensure compatibility with token limits of AI LLM APIs. The architecture of FIG. 2 supports distributed processing of video, imaging, and audio sections across multiple API calls to maximize efficiency and scalability.

Modules 200 are configured to: perform on-the-fly analysis of live data streams to detect anomalies, protocol deviations, and potential safety risks; and provide continuous updates delivered to the clinical team, ensuring immediate awareness of procedural issues. Modules 200 may also identify inefficiencies in procedural workflows based on multimodal data analysis, and provide recommendations to improve procedural efficiency and adherence to best practices.

Modules 200 may implement a scalable deployment system that supports both on-premise and cloud-based deployments, enabling scalability for various clinical settings. The token-aware segmentation and distributed analysis architecture allow the system to process large volumes of data efficiently.

Modules 200 may implement an adaptable protocol library. Modules 200 may incorporate customizable compliance protocols for different medical specialties and procedures. Modules 200 may allow for protocol updates to be dynamically applied without requiring system downtime.

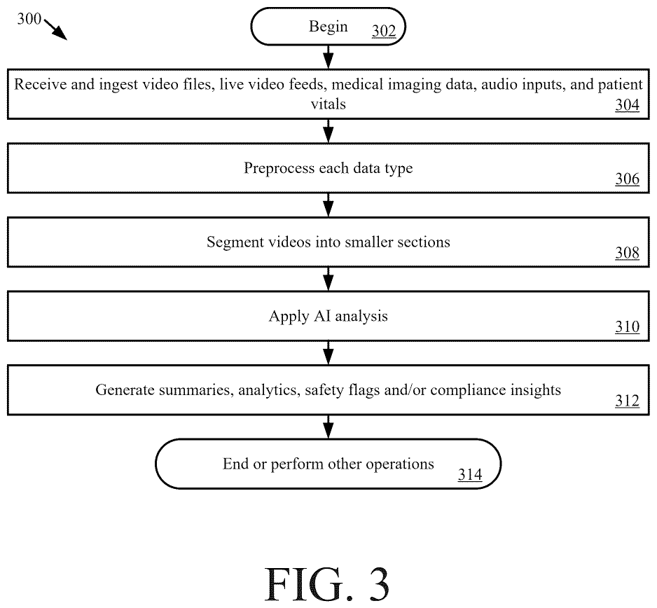

FIG. 3 provides a flow diagram of an illustrative method 300 for multimodal analysis. Method 300 begins at block 302 and continues to block 304 where a processor(s) (e.g., data ingestion module 202 of FIG. 2) receive(s), ingest(s) and/or synchronized multimodal data. The data ingestion may involve accepting and processing multimodal data including: video files in various formats; medical imaging data in formats such as DICOM, X-ray, MRI, and CT scans; audio inputs, including speech from clinicians and procedural sounds; and/or patient data, such as vitals and historical records, for integration with procedural insights. Block 204 may also involve (re)integrate analysis results from segmented data to form a comprehensive output.

Next in block 306, the processor(s) (e.g., pre-AI processing module 204 of FIG. 2) preprocess(es) each data type. The preprocessing operations can include, but are not limited to, video stabilization, imaging enhancement, audio noise reduction (or denoising), vital sign indexing, and/or keyframe extraction to improve analysis accuracy. Imaging preprocessing enhances resolution and extracts anatomical features. Audio preprocessing may include speech-to-text transcription, noise reduction, and synchronization with visual data. Temporal alignment synchronizes all data modalities, creating a unified timeline for AI analysis.

In block 308, the processor(s) (e.g., pre-AI processing module 204 of FIG. 1) segment(s) videos into smaller sections to enable efficient processing by AI large language model (LLM) APIs while maintaining contextual continuity. The video segmentation can involve: dividing video files or live video feeds into sections of a predefined length to adhere to the token limitations of AI LLM APIs; independently analyze each video section for key procedural elements; and/or recombining the video sections to create a coherent and comprehensive procedural summary. The segmentation may account for temporal overlaps to ensure that critical transitions between sections are captured and context is preserved.

In 310, the processor(s) (e.g., AI analysis engine 206 of FIG. 2) perform(s) AI analysis to identify procedural stages, instruments, anatomical structures, audio cues, and patient safety risks. The AI analysis may be partially performed by a computer vision submodule (e.g., computer vision submodule 230 of FIG. 2) to: detect procedural phases, instruments, and anatomical regions within video and imaging data; and/or recognize and analyze motion patterns, instrument usage, and transitions between procedural steps. The AI analysis may be partially performed by an audio processing submodule (e.g., audio processing submodule 232 of FIG. 2) to: transcribe speech from clinicians using natural language processing (NLP) techniques; and/or analyze contextual audio cues, such as alarms or procedural sounds, for additional insights. The AI analysis may be partially performed by a data integration submodule (e.g., data integration submodule 234 of FIG. 2) to: merge insights from video, imaging, audio, and patient data into a cohesive analysis; and/or reconstruct a complete procedural analysis by combining results from segmented video sections.

The AI analysis of block 310 may also involve an audio-visual synchronization process. This process can involve: aligning speech and sounds with corresponding video and imaging data; and contextualize real-time audio with visual and procedural information for enhanced situational awareness. Live video and audio feeds may be analyzed at regular intervals. The AI analysis of block 310 may further involve a patient-specific analysis that involves: integrating patient vitals and historical data into the procedural analysis; and generating personalized insights based on the patient's medical history and real-time physiological responses.

The processor(s) (e.g., output and report generation module 210 of FIG. 2) perform(s) operations in block 312 to generate summaries, analytics, compliance insights, and/or safety flags. The summaries may be generated by an output and report generation module (e.g., output and report generation module 210 of FIG. 2). The output and report generation module may generate procedural summaries in formats (e.g., Excel, Word, PDF, and/or JSON); detailed compliance reports, safety analyses, and graphical visualizations; and/or real-time updates during live procedures, including time-stamped safety alerts and trend analytics. The compliance insights may be provided by a real-time monitoring module (e.g., real-time monitoring module 208 of FIG. 2). In this regard, the real-time monitoring module may assess procedural compliance against predefined medical protocols, assess and/or flag safety risks (including extended procedural phases, irregular instrument usage, or abnormal vital sign trends). Alerts may also be generated and displayed in block 312 to the clinical team during live procedures, enabling immediate corrective action. Block 312 may also involve token-aware video processing in the processor(s) dynamically determine an optimal chunk size based on video complexity and content density, balancing token limits with analysis granularity. The safety alerts, procedural summaries, and compliance insights provide real-time feedback to the system. The real-time feedback may be used to, for example, control operations (autonomously or semi-autonomously) medical equipment 124 of FIG. 1 and/or robotic system(s) 132 of FIG. 1.

In block 312, the processor(s) may perform multi-phase procedural summary generation operations that involve: dividing procedures into distinct phases based on multimodal analysis; and/or summarizing each phase with key actions, time-stamped events, and compliance metrics. The processor(s) may perform robust report generation operations that involve: generating and outputting comprehensive reports in multiple formats, ensuring compatibility with EHR systems and external analytics platforms; and/or generating and outputting time-stamped procedural insights, safety metrics, and graphical representations of data trends.

Subsequently, method 300 continues to block 314 where it ends or other operations are performed. The other operations can include, but are not limited to, returning to block 302.

Method 300 may be used in various applications. These applications can include, but are not limited to, in vitro fertilization (IVF) procedures and/or monitoring, ultrasound imaging and/or guidance, endoscopy procedure and/or navigation assistance, angiography analysis, vascular risk assessment, surgical microscope integration, microsurgical precision assistance, virtual medical visit scribing, patient-specific context integration scenario, and/or virtual visit analytics.

In the IVF procedure scenario, method 300 may involve: ingesting video feeds from embryology labs (including microscopy and time-lapse imaging of embryo development); identifying key stages in embryo growth (such as fertilization, cleavage, and blastocyst formation); analyzing environmental data (e.g., incubator conditions, temperature) alongside video and imaging inputs; and/or generating and outputting a detailed timeline of embryo development with flagged anomalies or recommendations for optimal growth conditions. In the IVF monitoring scenario, method 300 may involve processing time-lapse microscopy images to predict embryo viability based on morphological and developmental criteria. Predictive analytics may also be generated to assist embryologists in selecting embryos for implantation.

In ultrasound imaging scenarios, method 300 may involve: processing live or recorded ultrasound images to detect anatomical structures, abnormalities, or physiological markers; integrating real-time audio commentary from clinicians with visual data for enhanced contextual understanding; and/or outputting annotated ultrasound images highlighting key areas of interest (such as fetal growth parameters, organ anomalies, or blood flow irregularities). In a real-time ultrasound guidance scenario, method 300 may involve: providing feedback during live imaging to assist clinicians in optimizing probe placement and image capture angles; and/or issuing alerts for out-of-range measurements, such as abnormal fetal heart rates or irregular blood flow patterns.

In endoscopy procedure scenarios, method 300 may involve: processing live video feeds from endoscopes to identify anatomical landmarks, abnormalities (e.g., polyps, ulcers), and procedural stages; tracking instrument movement and tissue interaction to ensure safety and procedural efficiency; and/or providing real-time compliance checks against standard endoscopic protocols, issuing alerts for deviations. In an endoscopic navigation assistance scenario, method 300 may involve: overlaying\s real-time annotations on the video feed to guide clinicians during complex procedures, such as navigating through the gastrointestinal tract; and/or summarizing the procedure with timestamps of key findings and recommendations for follow-up actions.

In an angiography analysis scenario, method 300 may involve: processing angiographic imaging data to detect vascular abnormalities, such as blockages, aneurysms, or stenosis; tracking contrast dye flow in real-time to assess vascular integrity and blood flow patterns; and/or outputting annotated angiograms with detailed analysis and recommendations for surgical or interventional procedures.

In an vascular risk assessment scenario, method 300 may involve: integrating angiographic findings with patient vitals and historical data to calculate risk scores for conditions such as myocardial infarction or stroke; and/or issuing alerts for critical vascular abnormalities requiring immediate intervention.

In surgical microscope integration scenarios, method 300 may involve: processing high-resolution video and imaging data captured through surgical microscopes; identifying microscopic anatomical features and tracks instrument-tissue interactions during microsurgery; and/or providing real-time safety alerts for unintended tissue damage or excessive instrument pressure. In microsurgical precision assistance scenarios, method 300 may involve: providing real-time guidance during delicate procedures (such as neurosurgery or ophthalmic surgery) by highlighting areas of interest and suggesting optimal instrument movements; and/or generating post-procedure reports with annotated images and compliance analytics.

In virtual medical visit scribing applications, method 300 may involve: processing live audio and video from telemedicine consultations to transcribe clinician-patient interactions; extracting key medical information (such as symptoms, diagnoses, and treatment plans); organizing the extracted key medical information into structured medical records; and/or outputting summaries in formats compatible with EHR systems, ensuring seamless documentation.

In patient-specific context integration scenario, method 300 may involve: incorporating historical medical records and real-time vitals data into virtual visit scribing; and/or flagging potential discrepancies or issues requiring further discussion based on the patient's medical history and current symptoms.

In virtual visit analytics scenarios, method 300 may involve: tracking key metrics from virtual consultations (such as average consultation time, most common diagnoses, and medication trends); and/or providing feedback to clinicians on conversational clarity, adherence to clinical guidelines, and patient engagement.

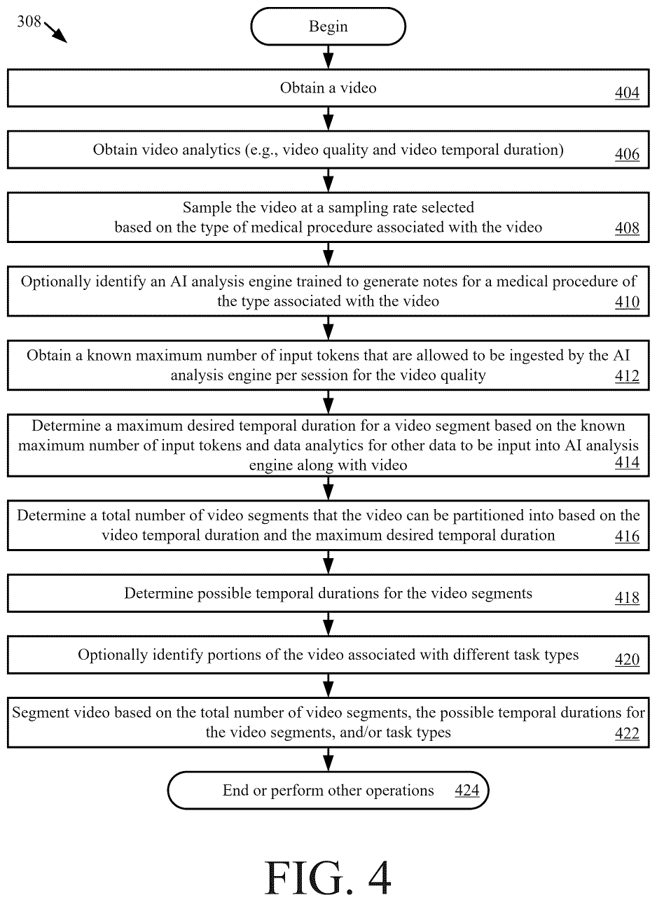

FIG. 4 provides a flow diagram of operations that may be performed in block 308 of FIG. 3 to segment a video. The process of FIG. 4 can include more or less operations than that shown for any given application. The operation of FIG. 4 can be in the same or different order as that shown for any given application.

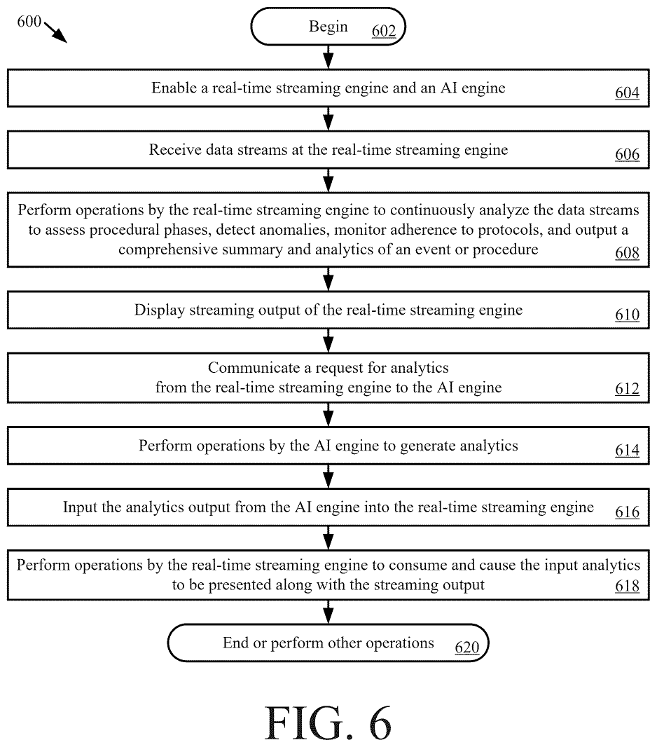

Some or all of the operations of FIG. 4 may be performed by a system (e.g., system 100 of FIG. 1), processor (e.g., central processing unit 606 of FIG. 6) and/or computing device (e.g., computing device 118 of FIG. 1). The process of FIG. 4 will be described in relation to a processor for ease of illustration. The video segments may then be provided to AI engine(s) (e.g., AI engine(s) 120 of FIG. 1 and/or AI analysis engine(s) 206 of FIG. 2) to be processed in series or in parallel for generating electronic medical procedure notes.

The operations comprise obtaining a video in block 404 and video analytics in block 406. The video analytics can include, but are not limited to, video quality (e.g., 1080p or 4K resolution), video length or temporal duration, and/or an associated type of medical procedure. In block 408, the video is sampled at a sampling rate selected or otherwise obtained based on the type of medical procedure associated with the video. The sampling rate may be defined a N frames for every M seconds. Each of N and M is an integer equal to or greater than one. N and/or M may be obtained, determined, computed or selected based on the type of medical procedure associated with the video. For example, a sampling rate of one frame per one second is selected when the medical procedure is an ACL repair. A sampling rate of five frames per one second is selected when the medical procedure is retinal surgery. The present solution is not limited to the particulars of this example. Any known or to be known video sampling technique may be used here.

In optional block 410, the processor identifies an AI analysis engine from a plurality of AI analysis engines that is trained to generate notes for a medical procedure of the type associated with the video. The processor obtains, in block 412, a known maximum number of input tokens that are allowed to be ingested by the AI analysis engine per session for the video quality. For example, the maximum number of input tokens may roughly equate to eighteen minutes of 1080p video by taking one frame for every one second of video. The present solution is not limited by the particulars of this example.

In block 414, the processor determines a maximum desired temporal duration for a video segment based on the known maximum number of input tokens and data analytics for other data to be input into AI analysis engine along with video. For example, an amount of input tokens for other data equates to around eight minutes of video. So, a maximum of ten minutes of video is allowed per session (i.e., 18 minutes−8 minutes=10 minutes). The present solution is not limited to the particulars of this example.

In block 416, the processor determines a total number of video segments that the video can be partitioned into based on the video temporal duration and the maximum desired temporal duration. For example, the video is forty-five minutes long and the maximum desired temporal duration for a video segment is ten minutes. Thus, the video may be partitioned into five segments (i.e., 45/10=4.5 rounded to 5). The present solution is not limited to the particulars of this example.

In block 418, the processor determines possible temporal durations for the video segments. For example, the video is forty-five minutes long and the maximum desired temporal duration for a video segment is ten minutes. Thus, the video may be partitioned into five segments (i.e., 45/10=4.5 rounded to 5). Four of the segments may be ten minutes in duration, while the fifth segment may be five minutes in duration (i.e., 10 minutes, 10 minutes, 10 minutes, 10 minutes, 5 minutes). The present solution is not limited to the particulars of this example.

In optional block 420, the processor identifies portions of the video associated with different tasks. For example, the video may contain a first portion including pre-operative tasks (e.g., tasks to prepare for medical procedure), a second portion including operative tasks (e.g., tasks to perform medical procedure), and a third portion including post-operative tasks (e.g., tasks to perform post-operative care and/or operating room cleaning). The present solution is not limited to the particulars of this example.

In block 422, the processor performs operations to partition the video into segments based on the total number of video segments determined in block 416, the possible temporal durations of the video segments determined in block 418, and/or the different task types associated with the portions of video identified in block 420. For example, the video is segmented such that a first segment including operative tasks has a temporal duration of five minutes and each of the four remaining segments have a temporal duration of ten minutes. The present solution is not limited to the particulars of this example.

Subsequently, the process continues to block 424 where it ends or other operations are performed. The other operations can include, but are not limited to, returning to block 404.

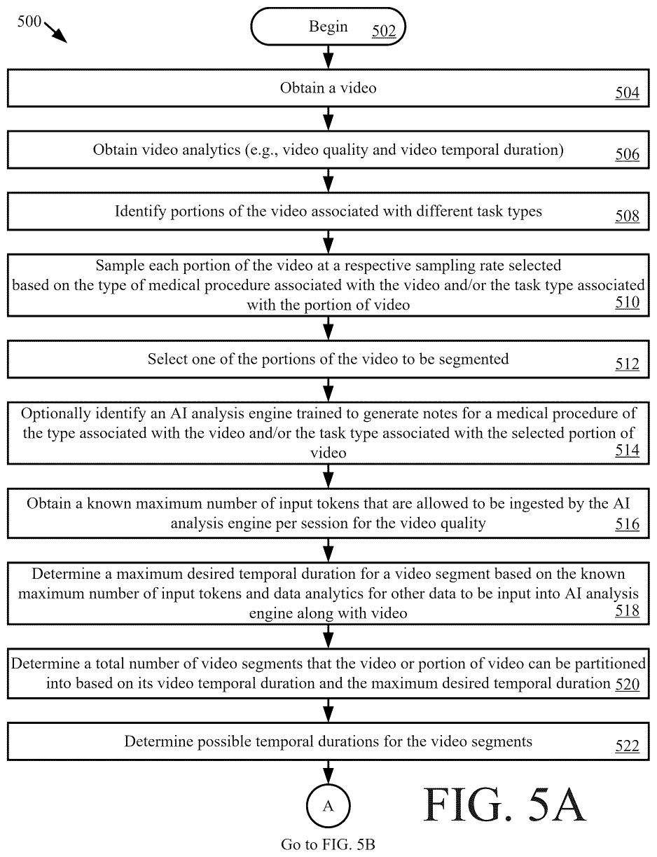

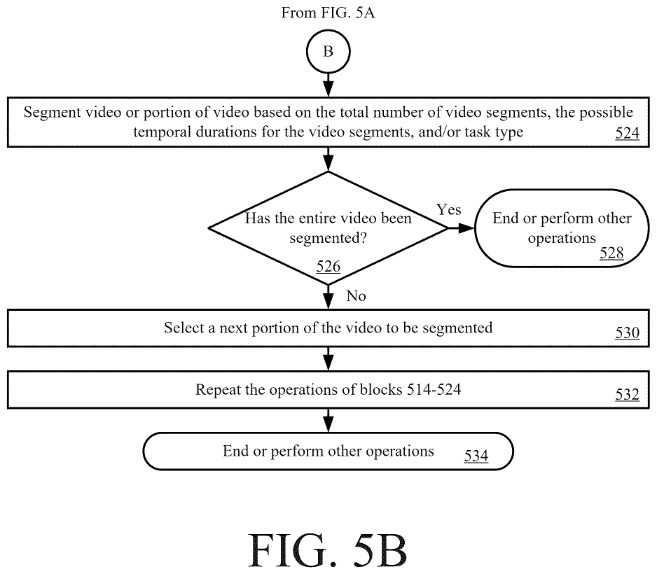

FIG. 5 provides a flow diagram of operations that may be performed in block 308 of FIG. 3 to segment a video. The process of FIG. 5 can include more or less operations than that shown for any given application. The operation of FIG. 5 can be in the same or different order as that shown for any given application.

Some or all of the operations of FIG. 5 may be performed by a processor (e.g., central processing unit 606 of FIG. 6) and/or computing device (e.g., computing device 118 of FIG. 1). The video segments may then be provided to AI engine(s) (e.g., AI engine(s) 120 of FIG. 1 and/or AI analysis engine(s) 206 of FIG. 2) to be processing in series or in parallel for generating electronic medical procedure notes.

Method 500 begins at block 502 and continues to blocks 504-506 where the processor obtains a video and video analytics. The video analytics can include, but are not limited to, video quality (e.g., 1080p or 4K resolution), video length or temporal duration, and/or an associated type of medical procedure.

In block 508, the processor identifies portions of the video associated with different tasks. For example, the video may contain a first portion including pre-operative tasks (e.g., tasks to prepare for medical procedure), a second portion including operative tasks (e.g., tasks to perform medical procedure), and a third portion including post-operative tasks (e.g., tasks to perform post-operative care and/or operating room cleaning). The present solution is not limited to the particulars of this example.

Next in block 510, the processor samples each portion of the video at a respective sampling rate. The respective sampling rate may be selected based on the type of medical procedure associated with the video and/or the task type associated with the portion of video. For example, a first sampling rate is selected when the medical procedure is an ACL repair and/or the first portion of video includes pre-operative tasks. A different second sampling rate is selected when the medical procedure is an ACL repair and/or the second portion of video includes operative tasks of any type or of specific types. A third sampling rate is selected when the medical procedure is an ACL repair and/or the third portion of video includes post-operative tasks. The third sampling rate may be the same as or different than the first sampling rate and/or the second sampling rate. The present solution is not limited to the particulars of this example.

In block 512, the processor selects one of the portions of video to be segmented. The processor may optionally identify an AI analysis engine trained to generate notes for a medical procedure of the type associated with the video and/or the task type associated with the selected portion of video, as shown by optional block 514.

The processor obtains, in block 516, a known maximum number of input tokens that are allowed to be ingested by the AI analysis engine per session for the video quality. For example, the maximum number of input tokens may roughly equate to eighteen minutes of 1080p video by taking one frame for every one second of video. The present solution is not limited by the particulars of this example.

The processor may determine or otherwise obtain, in block 518, a known maximum number of input tokens that are allowed to be ingested by the AI analysis engine per session for the video quality. For example, the maximum number of input tokens may roughly equate to eighteen minutes of 1080p video by taking one frame for every one second of video. The present solution is not limited by the particulars of this example.

In 520, the processor determines a total number of video segments that the video or portion of video can be partitioned into based on its video temporal duration and the maximum desired temporal duration. For example, the video or portion of video is forty-five minutes long and the maximum desired temporal duration for a video segment is ten minutes. Thus, the video or portion of video may be partitioned into five segments (i.e., 45/10=4.5 rounded to 5). The present solution is not limited to the particulars of this example.

In 522, the processor determines possible temporal durations for the video segments. For example, the video is forty-five minutes long and the maximum desired temporal duration for a video segment is ten minutes. Thus, the video or portion of video may be partitioned into five segments (i.e., 45/10=4.5 rounded to 5). Four of the segments may be ten minutes in duration, while the fifth segment may be five minutes in duration (i.e., 10 minutes, 10 minutes, 10 minutes, 10 minutes, 5 minutes). The present solution is not limited to the particulars of this example.

Upon completing the operations of block 522, method 500 continues to block 524 of FIG. 5B. As shown in FIG. 5B, block 524 involves segmenting the video or portion of video based on the total number of video segments determined in block 520, the possible temporal durations for the video segments determined in block 522, and/or associated task type.

Next, the processor determines whether the entire video has been segments. If so [526:YES], method 500 continues to block 528 where it ends or other processing is performed (e.g., return to block 502 of FIG. 5A).

Otherwise [526:NO], method 500 continues to block 530 where a next portion of the video is selected to be segmented. The processor repeats the operations of blocks 514-526 to segment the next portion of the video, as shown by block 532. In the multi-video portion scenario, the video may be segmented into a plurality of segments having the same or different temporal durations and/or the same or different sampling rates. For example, a video has a length or temporal duration of forty-five minutes and three portions associated with different task types (e.g., pre-operative, operative, and post-operative). The video is segmented into five segments: a first segment having a temporal duration of five minutes and a relatively high sampling rate; a second segment having a temporal duration of ten minutes and a relatively low sampling rate; a third segment having a temporal duration of ten minutes and a relatively low sampling rate; a fourth segment having a temporal duration of ten minutes and a relatively low sampling rate; and a fifth segment having a temporal duration of ten minutes and a relatively low sampling rate. The present solution is not limited to the particulars of this example.

Upon completing the operations of block 532, method 500 continues to block 534 where it ends or other operations are performed. The other operations can include, but are not limited to, returning to block 502 of FIG. 5A.

There are maximum token limits for intelligent processing that may be less than the maximum token limit the LLMs allow for. For example, if the AI analysis engine(s) may be prompted to output major steps from a ten minute procedure, then it will do so with complete accuracy and completeness. But if the AI analysis engine(s) are asked to output major steps from a thirty minute procedure, it may miss certain steps or not output all of the steps with high accuracy. Therefore, the processor may break down a thirty minute video into multiple chunks to output major steps with high accuracy, even though the thirty minute video is within the LLM token limit. In this case, method(s) 400, 500 may be modified accordingly.

FIG. 6 provides a flow diagram of another method 600 for operating a system (e.g., system 100 of FIG. 1). Method 600 begins with block 602 and continues to block 604 where a real-time streaming engine (e.g., real-time streaming engine 126 of FIG. 1) and an AI engine (e.g., AI engine 120 of FIG. 1) are enabled. In block 606, data stream(s) is(are) received at the real-time streaming engine. The real-time streaming engine performs operations in block 608 to continuously analyze the data stream(s) to assess procedural phases, detect anomalies, monitor adherence to protocols, and output a comprehensive summary and analytics of an event or procedure. The streaming output of the real-time streaming engine is displayed on a display of a computing device (e.g., computing device 118 of FIG. 1).

In block 612, the real-time streaming engine communicates a request for analytics to the AI engine. The AI engine performs operations in next block 614 to generate the requested analytics. The analytics output from the AI engine are input into the real-time streaming engine along with the data streams, as shown by block 616. The real-time streaming engine performs operations in block 618 to consume the inputs and cause the analytics to be presented along with the streaming output (e.g., streaming video). Subsequently, method 600 continues to block 620 where it ends or other operations are performed. The other operations can include, but are not limited to, returning to block 602.

For example, in a surgery context, the real-time streaming engine (e.g., streaming API) collects data (e.g., video, audio or other data), performs an algorithm, and causes the display of a live stream output on the computing device. The AI engine (e.g., LLM API) is run in the background. The output of the AI engine (e.g., LLM API) is provided as an input to the real-time streaming engine(s) (e.g., streaming API). The real-time streaming engine(s) (e.g., streaming API) use(s) this input to generate its output. In this case, the user may be presented, for example, a live streaming video, audio or other data with the LLM output, context or content overlaid on the live streaming video.

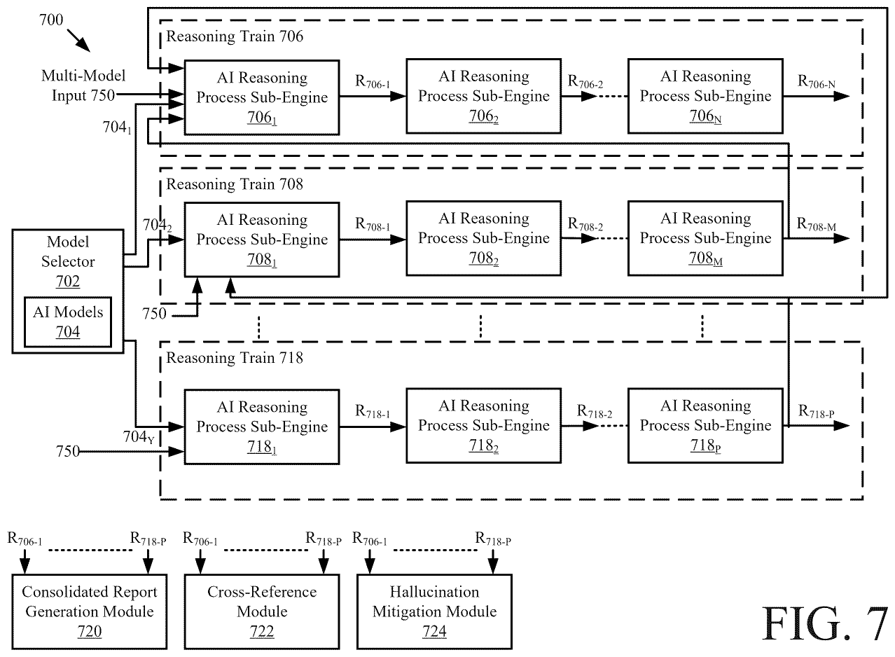

In some scenarios, AI engine(s) 120 of FIG. 1 and/or AI analysis engine(s) 206 of FIG. 2 implement(s) a cascaded reasoning train architecture. A block diagram of an illustrative cascaded reasoning train architecture 700 is shown in FIG. 7.

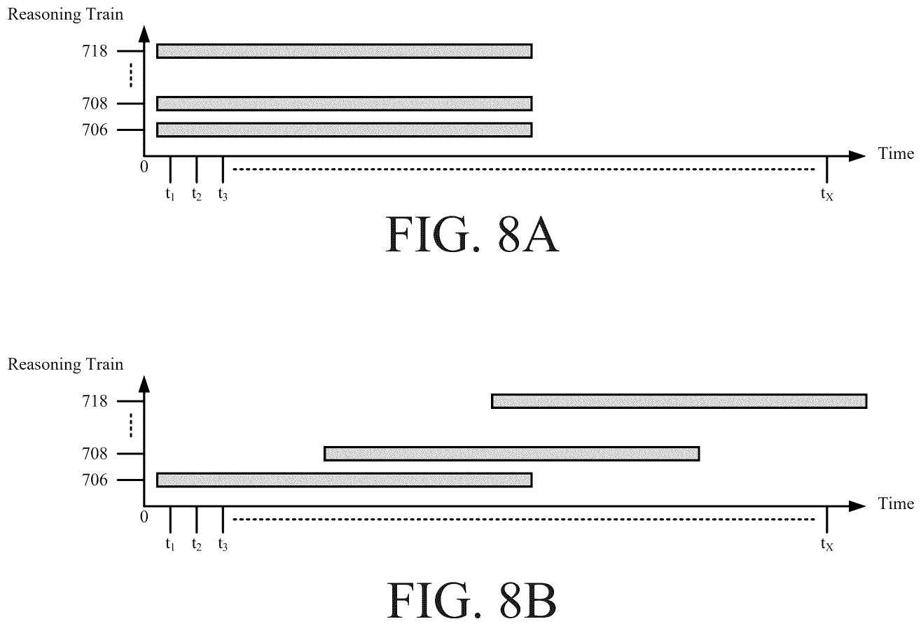

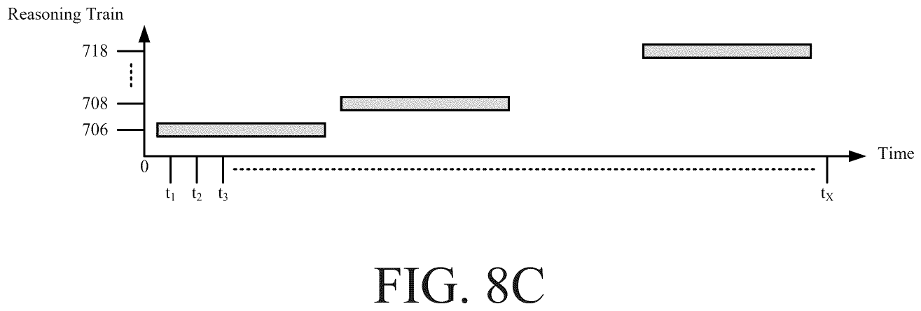

Architecture 700 comprises a plurality of independently executing reasoning trains 706, 708, . . . , 718. The reasoning trains 706, 708, . . . , 718 may be concurrently or simultaneously executing the same or different reasoning processes. Each reasoning train 706, 708, . . . , 718 may be considered a train of thought for AI reasoning. The reasoning trains 706, 708, . . . , 718 may operate at different temporal cadences and/or different computational depths. Each reasoning train may operate at a unique cadence. The unique cadence can include, but is not limited to, seconds-level intervals, minute-level intervals, hour-level intervals, and/or dynamically calculated intervals based on procedural phase, detected anomalies, system load, and/or risk thresholds. For example, in some scenarios, reasoning train 706 runs every size seconds, while reasoning train 708 runs every six minutes and reasoning train 718 runs every five minutes. The present solution is not limited to the particulars of this example. The reasoning trains 706, 708, . . . , 718 may execute in parallel as shown in FIG. 8A, partially overlapping time windows as shown in FIG. 8B, and/or staggered time schedules or windows as shown in FIG. 8C.

Each reasoning train 706, 708, . . . , 718 may: (i) execute at a predefined, dynamically adjustable, event-triggered, and/or threshold-triggered time interval; (ii) utilize an AI model selected from a plurality of available AI models 704; (iii) generate structured intermediate outputs; (iv) maintains an associated memory state; and/or (v) provide outputs to other reasoning trains and to a consolidated report generation module 720. Any number of reasoning trains can be utilized in accordance with a given application. Certain ones of the reasoning trains may function as temporal memory layers, including short-term memory aggregation and long-term longitudinal trend analysis across procedural intervals.

Each reasoning train 706, 708, . . . , 718 may utilize any selected AI model. The AI model comprises a computer program trained to recognize patterns, make predictions, and/or generate new content based on multi-model medical data and/or other information. The AI model can include, but is not limited to, low-latency models, high-capability models, multimodal models, domain-specialized models, lightweight inference models, large parameter models, successor models, and/or other computer program models that do not require constant human guidance. The AI model for each reasoning train may be selected by a model selector 702 from a plurality of different AI models 704. For example, model selector 702 may select AI model 704₁ and provide the same to reasoning train 706 for use during its reasoning process. Model selector 702 may select AI model 704₂ and provide the same to reasoning train 708 for use during its reasoning process. Model selector 702 may select AI model 704_{Y} and provide the same to reasoning train 718 for use during its reasoning process. Model selector 702 may dynamically switch, replace, upgrade, and/or reallocate AI models 704 used by any reasoning train 706, 708, . . . , 718 without altering architecture 700. Model selector 702 may select AI models 704 based on latency requirements, computational constraints, procedural complexity, anomaly detection thresholds, confidence scores, and/or user-defined parameters. The architecture 700 is not limited to any specific model vendor, parameter size, token limit, or model class.

As noted above, each reasoning train 706, 708, . . . , 718 may generate structured intermediate outputs. In this regard, each reasoning train 706, 708, . . . , 718 may comprise a plurality of AI reasoning process sub-engines that each generate an output. For example, reasoning train 706 comprises cascaded AI reasoning process sub-engines 706₁, 706₂, . . . , 706_{N} that generate outputs R₇₀₆₋₁, R₇₀₆₋₂, . . . , R_{706-N}. Reasoning train 708 comprises cascaded AI reasoning process sub-engines 708₁, 708₂, . . . , 708_{M} that generate outputs R₇₀₈₋₁, R₇₀₈₋₂, . . . , R_{708-M}. Reasoning train 718 comprises cascaded AI reasoning process sub-engines 718₁, 718₂, . . . , 718_{P-1} that generate outputs R₇₁₈₋₁, R₇₁₈₋₂, . . . , R_{718-P}. M, N and P are integers. M, N and P may be the same as or different than each other. The outputs R₇₀₆₋₁, R₇₀₆₋₂, . . . , R_{706-N}, R₇₀₈₋₁, R₇₀₈₋₂, . . . , R_{708-M}, R₇₁₈₋₁, R₇₁₈₋₂, . . . , R_{718-P} can include, but are not limited to, detected procedural phases, instrument identifications, safety alerts, compliance assessments, risk scores, longitudinal trends, and/or other analytical conclusions.

In some scenarios, the reasoning trains 706, 708, . . . , 718 may operate within a cascading context architecture. Each reasoning train may receive, within its context window, structured outputs generated by one or more other reasoning trains during prior temporal intervals. For example, reasoning trains 708 and 718 may provide their intermediate outputs R₇₀₈₋₁, R₇₀₈₋₂, . . . , R_{708-M-1}, R₇₁₈₋₁, R₇₁₈₋₂, . . . , R_{718-P-1} and/or final output R_{708-M}, R_{718-P} to reasoning train 706 for use during a next temporal interval. As reasoning train 706 generates new outputs in the next temporal interval, it may incorporate validated outputs from the other reasoning train(s) 708, 718 as contextual background information. This cascading mechanism allows each reasoning train to maintain awareness of prior analytical conclusions, detected anomalies, compliance findings, safety alerts, memory-layer outputs, and evolving procedural context. Context propagation between reasoning trains may be filtered, validated, summarized, weighted, or token-optimized prior to insertion into a receiving train's context window. The cascading process may include temporal tagging, confidence scoring, and selective inclusion based on procedural phase or anomaly thresholds.

Outputs of the two or more reasoning trains 706, 708, . . . , 718 may be compared to each other by a cross-reference module 722. Cross-reference module 722 may be configured to: compare semantic equivalence across outputs R₇₀₆₋₁, R₇₀₆₋₂, . . . , R_{706-N}, R₇₀₈₋₁, R₇₀₈₋₂, . . . , R_{708-M}, R₇₁₈₋₁, R₇₁₈₋₂, . . . , and/or R_{718-P}; detect inconsistencies or contradictions in the outputs R₇₀₆₋₁, R₇₀₆₋₂, . . . , R_{706-N}, R₇₀₈₋₁, R₇₀₈₋₂, . . . , R_{708-M}, R₇₁₈₋₁, R₇₁₈₋₂, . . . , and/or R_{718-P}; identify anomalous outputs produced by a subset of reasoning trains; assign confidence weights to each reasoning train; perform majority-based, weighted, rule-based, or adaptive consensus validation of outputs R₇₀₆₋₁, R₇₀₆₋₂, . . . , R_{706-N}, R₇₀₈₋₁, R₇₀₈₋₂, . . . , R_{708-M}, R₇₁₈₋₁, R₇₁₈₋₂, . . . , and/or R_{718-P}; and/or trigger or otherwise initiate re-analysis by the reasoning train(s) when discrepancies exceed a defined threshold.

A hallucination mitigation module 724 may be provided to optionally determine if a hallucination has or has not occurred. A hallucination may be considered to have occurred when a reasoning train produces a conclusion inconsistent with outputs from other reasoning trains analyzing the same multimodal inputs. Outputs of the reasoning train associated with the hallucination may be suppressed, annotated, down-weighted, escalated for deeper analysis, and/or flagged for human review prior to inclusion in a final consolidated report.

Outputs of the reasoning trains 706, 708, . . . , 718 may be provided to consolidated generation module 720. Consolidated report generation module 720 may be configured to integrate validated, cross-referenced, and/or cascaded outputs R₇₀₆₋₁, R₇₀₆₋₂, . . . , R_{706-N}, R₇₀₈₋₁, R₇₀₈₋₂, . . . , R_{708-M}, R₇₁₈₋₁, R₇₁₈₋₂, . . . , and/or R_{718-P} from the reasoning trains to produce a final procedural report. The final procedural report may reflect integrated contributions from multiple reasoning trains. The final procedural report may also incorporate confidence scores, validation indicators, temporal correlations, compliance findings, safety alerts, longitudinal trends, and/or contextual insights derived from cascading memory layers.

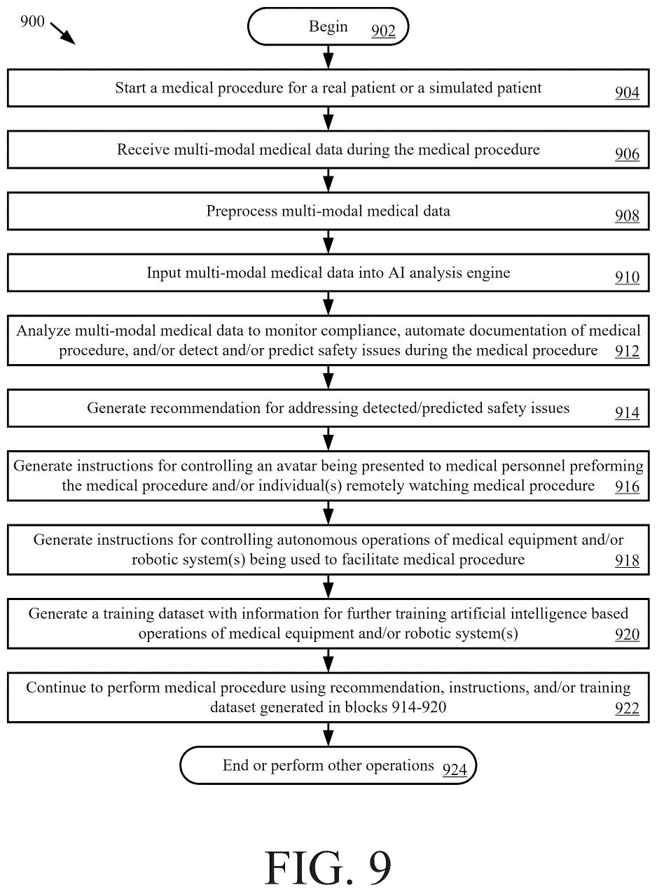

FIG. 9 provides a flow diagram of an illustrative method 900 for operating a medical system (e.g., system 100 of FIG. 1) in accordance with the present solution. Method 900 may include more or fewer operations than shown. The operations of method 900 may be performed in the same or different order than that shown.

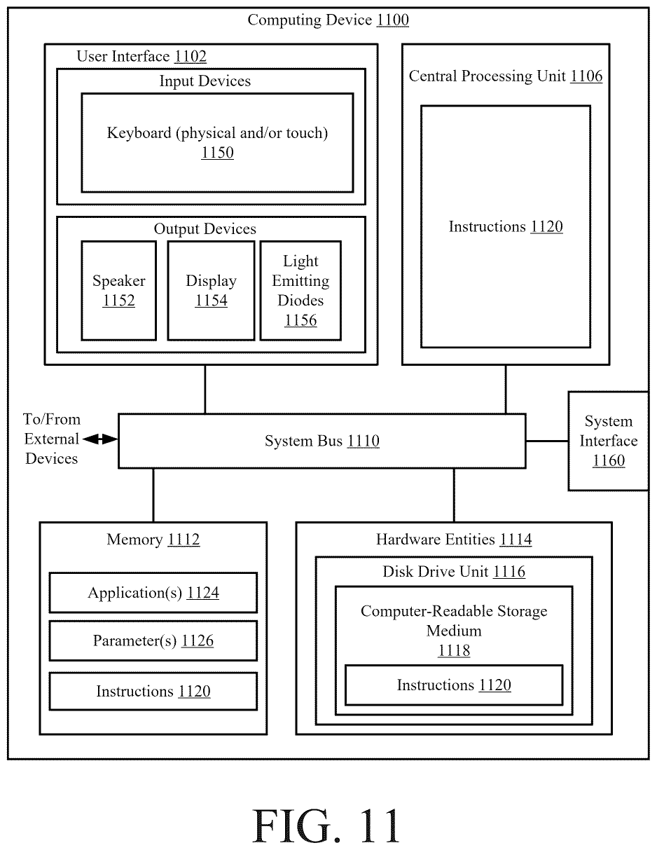

Method 900 begins at block 902 and continues to block 904 where a medical procedure is started for a real patient or a simulated patient. During the medical procedure, multi-modal medical data is captured and/or collected by multimodal input sources (e.g., multimodal input sources 102 of FIG. 1). The multi-modal medical data is received in block 906 by a processor or computing device (e.g., computing device(s) 118 of FIG. 1, server(s) 114 of FIG. 1, and/or central processing unit 1106 of FIG. 11). The multimodal medical data is pre-processed in block 908. The pre-processing may include, but is not limited to, the pre-processing described above in relation to blocks 302-308 of FIG. 3.

The pre-processed multi-modal medical data is input into AI engine(s) (e.g., AI engines 120 of FIG. 1) as shown by block 910. The AI engine(s) analyze(s) the input multi-modal medical data to monitor compliance, automate documentation of medical procedure, and/or detect and/or predict safety issues during the medical procedure. This analysis may include, but is not limited to, the analysis described above in relation to blocks 310-312 of FIG. 3, the analysis described above in relation to block 608 of FIG. 6, and/or the analysis described in any other section of this document. Based on results of this analysis, the AI engine(s) generates output(s) in blocks 914-920. The output(s) can include, but is(are) not limited to, a recommendation for addressing detected/predicted safety issues, instructions for controlling an avatar being presented to medical personnel preforming the medical procedure and/or individual(s) remotely watching medical procedure, instructions for controlling autonomous operations of medical equipment and/or robotic system(s) being used to facilitate medical procedure, and/or a training dataset with information for further training artificial intelligence based operations of medical equipment and/or robotic system(s).

The medical procedure is continued to be performed using the output(s) of the AI engine(s) as shown by block 922. Block 922 may involve, for example: updating an electronic health record for the patient to improve care coordination amongst a plurality of medical professionals and/or enhance patient safety in medical care and/or medication distribution; controlling autonomous operations of an avatar (e.g., avatar engine 130 of FIG. 1); and/or controlling autonomous operations of a robotic system (e.g., medical equipment 124 of FIG. 1, and/or robotic system 132 of FIG. 1). The autonomous operations of the avatar that are controlled can include, but are not limited to, assisting a user with inquiries, providing information to the user in response to the inquiries, auditorily presenting information output from the AI analysis engine, and/or responding to commands from the user. Any known or to be known technique for controlling an avatar can be used here. The autonomous operations of the robotic system that are controlled can include, but are not limited to, navigating a medical tool relative to an anatomy of a patient. Any known or to be known technique for robotically navigating a tool can be used here.

Subsequently, method 900 continues with block 924 where it ends or other operations are performed. The other operations include, but are not limited to, returning to block 906, completing the medical procedure, and/or further training the artificial intelligence based operations of medical equipment and/or robotic system(s).

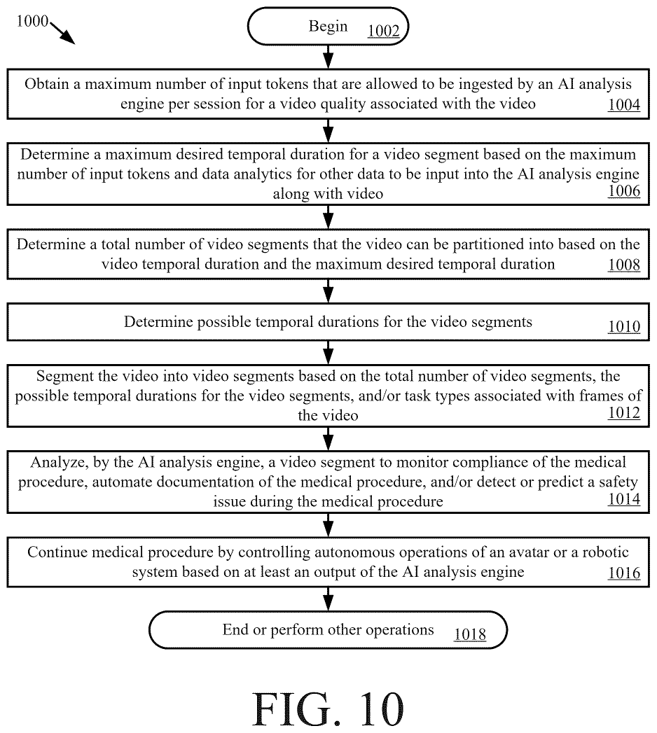

FIG. 10 provides a flow diagram of an illustrative method 1000 for processing and using video of a medical procedure in progress. Method 1000 can include more or fewer operations than that shown. The operations of method 1000 may be performed in the same or different order than that shown.

Method 1000 begins at block 1002 and continues to block 1004 where a processor (e.g., computing device(s) 118 of FIG. 1, server(s) 114 of FIG. 1, and/or central processing unit 1106 of FIG. 11) performs operations to: optionally identify an AI analysis engine from a plurality of AI analysis engines trained to generate notes for a medical procedure of a type associated with the video; and/or obtains a maximum number of input tokens that are allowed to be ingested by the AI analysis engine (e.g., AI engine(s) 120 of FIG. 1, AI analysis engine 206 of FIG. 2, and/or AI analysis engine 700 of FIG. 7) per session for a video quality associated with the video. The AI analysis engine may optionally have a cascaded reasoning train architecture (e.g., architecture 700 of FIG. 7).

Next in block 1006, the processor performs operations to determine a maximum desired temporal duration for a video segment based on the maximum number of input tokens and data analytics for other data to be input into the AI analysis engine along with video. The other data and video may be temporally correlated with each other via timestamps linking segments of video to detected events that occurred during the medical procedure. The processor also performs operations in block 1008 to determine a total number of video segments that the video can be partitioned into based on the video temporal duration and the maximum desired temporal duration. In block 1010, the processor determines possible temporal durations for the video segments. The processor segments the video in block 1012 into a plurality of video segments based on the total number of video segments, the possible temporal durations for the video segments, and/or task types associated with frames of the video.

The AI analysis engine analyzes a video segment in block 1014 to monitor compliance of the medical procedure, automate documentation of the medical procedure, and/or detect or predict a safety issue during the medical procedure. In some scenarios, block 1014 may also involve: selecting a sampling rate for the video based on a type of medical procedure associated with the video; and/or generating by the AI analysis engine one or more of a recommendation for addressing a detected or predicted safety issue associated with the medical procedure, instructions for controlling the autonomous operations of the avatar or robotic system to facilitate the medical procedure, and a training dataset for further training artificial intelligence operations of the avatar or the robotic system.

The medical procedure is continued by controlling autonomous operations of an avatar (e.g., avatar engine(s) 130 of FIG. 1) or a robotic system (e.g., medical equipment 124 of FIG. 1, and/or autonomous robotic system(s) 132 of FIG. 1) based on at least an output of the AI analysis engine, as shown by block 1016. The autonomous operations of the avatar that may be controlled include, but are not limited to, assisting a user with inquiries, providing information to the user in response to the inquiries, auditorily presenting information output from the AI analysis engine, and/or responding to commands from the user. The autonomous operations of the robotic system that may be controlled include, but are not limited to, navigating a medical tool relative to an anatomy of a patient. The medical tool can include, but is not limited to, any known or to be known autonomous staple device, autonomous suture device, smart catheter, smart ablation device, autonomous surgical system, and/or autonomous endoscope. The autonomous surgical system can comprise a waterjet, hair transplantation device, bone preparation device, imaging systems, scanning device, minimally invasive surgical device, insulin pumps, implantable cardioverter-defibrillators, and/or wearable device. Control of the autonomous operations of the avatar or the robotic system may be further based on one or more of data received from a wearable device worn (e.g., other device(s) 150 of FIG. 1) by a medical professional facilitating the medical procedure, data received from an invasive or non-invasive brain-interface device (e.g., other device(s) 150 of FIG. 1) associated with the medical professional facilitating the medical procedure, and data received from a electro-mechanical control device (e.g., other device(s) 150 of FIG. 1) actuated by the medical professional facilitating the medical procedure. The wearable device can include, but is not limited to, a smart watch, and/or a headset. The invasive or non-invasive brain-interface device can include, but is not limited to, a brain-computer interface (BCI). The electro-mechanical control device can include, but is not limited to, a hand-actuated controller and/or a hands-fee controller. The hand-actuated controller can include a depressible button and/or joy stick. The hands-free controller can include, but is not limited to, a foot peddle.

Subsequently, method 1000 continues to block 1002 where it ends or other operations are performed. The other operations can include, but are not limited to, dynamically changing an artificial intelligence model being used by the AI analysis engine during the medical procedure without altering the cascaded reasoning train architecture, operation(s) of method 300 of FIG. 3, operation(s) of method 500 of FIG. 5, operation(s) of method 600 of FIG. 6, and/or operation(s) of method 900 of FIG. 9.

Referring now to FIG. 11, there is shown an illustrative architecture for a computing device 1100. Components 102, 114, 118, 120, 124 of FIG. 1 and/or module(s) 202, 204, 206, 208, 210 of FIG. 2 is/are the same as or similar to computing device 1100. As such, the discussion of computing device 1100 is sufficient for understanding the listed components of FIG. 1.

Computing device 1100 may include more or less components than those shown in FIG. 11. However, the components shown are sufficient to disclose an illustrative solution implementing the present solution. The hardware architecture of FIG. 11 represents one implementation of a representative computing device configured to receive information, process the receive information, transmit information and/or control operations of one or more robots, as described herein. As such, the computing device 1100 of FIG. 11 implements at least a portion of the method(s) described herein.

Some or all components of the computing device 1100 can be implemented as hardware, software and/or a combination of hardware and software. The hardware includes, but is not limited to, one or more electronic circuits. The electronic circuits can include, but are not limited to, passive components (e.g., resistors and capacitors) and/or active components (e.g., amplifiers and/or microprocessors). The passive and/or active components can be adapted to, arranged to and/or programmed to perform one or more of the methodologies, procedures, or functions described herein.

As shown in FIG. 11, the computing device 1100 comprises a user interface 1102, a Central Processing Unit (CPU) 1106, a system bus 1110, a memory 1112 connected to and accessible by other portions of computing device 1100 through system bus 1110, a system interface 1160, and hardware entities 1114 connected to system bus 1110. The user interface can include input devices and output devices, which facilitate user-software interactions for controlling operations of the computing device 1100. The input devices include, but are not limited to, a physical and/or touch keyboard 1150. The input devices can be connected to the computing device 1100 via a wired or wireless connection (e.g., a Bluetooth® connection). The output devices include, but are not limited to, a speaker 1152, a display 1154, and/or light emitting diodes 1156. System interface 1160 is configured to facilitate wired or wireless communications to and from external devices (e.g., network nodes such as access points, etc.).

At least some of the hardware entities 1114 perform actions involving access to and use of memory 1112, which can be a random access memory (RAM), a disk drive, flash memory, a universal serial bus (USB) drive and/or another hardware device that is capable of storing instructions and data. Hardware entities 1114 can include a disk drive unit 1116 comprising a computer-readable storage medium 1118 on which is stored one or more sets of instructions 1120 (e.g., software code) configured to implement one or more of the methodologies, procedures, or functions described herein. Instructions 1120 can also reside, completely or at least partially, within the memory 1112 and/or within the CPU 1106 during execution thereof by the computing device 1100. The memory 1112 and the CPU 1106 also can constitute machine-readable media. The term “machine-readable media”, as used here, refers to a single medium or multiple media (e.g., a centralized or distributed database, and/or associated caches and servers) that store the one or more sets of instructions 1120. The term “machine-readable media”, as used here, also refers to any medium that is capable of storing, encoding or carrying a set of instructions 1120 for execution by the computing device 1100 and that cause the computing device 1100 to perform any one or more of the methodologies of the present disclosure.

Without excluding further possible embodiments, certain example embodiments are summarized in the following clauses.
Clause 1: A method for processing and using video of a medical procedure in progress, comprising: obtaining, by a processor, a maximum number of input tokens that are allowed to be ingested by an artificial intelligence (AI) analysis engine per session for a video quality associated with the video; determining, by the processor, a maximum desired temporal duration for a video segment based on the maximum number of input tokens and data analytics for other data to be input into the AI analysis engine along with video; determining, by the processor, a total number of video segments that the video can be partitioned into based on the video temporal duration and the maximum desired temporal duration; determining, by the processor, possible temporal durations for the video segments; segmenting, by the processor, the video into a plurality of video segments based on the total number of video segments, the possible temporal durations for the video segments, and/or task types associated with frames of the video; analyzing, by the AI analysis engine, a video segment of the plurality of video segments to monitor compliance of the medical procedure, automate documentation of the medical procedure, and/or detect or predict a safety issue during the medical procedure; and continuing the medical procedure by controlling autonomous operations of an avatar or a robotic system based on at least an output of the AI analysis engine.
Clause 2: The method according to clause 1, further comprising selecting a sampling rate for the video based on a type of medical procedure associated with the video.
Clause 3: The method according to any preceding clause, further comprising identifying the AI analysis engine from a plurality of AI analysis engines trained to generate notes for a medical procedure of a type associated with the video.
Clause 4: The method according to any preceding clause, further comprising providing the video segment of the plurality of video segments to the AI analysis engine for use in generating the medical procedure notes.
Clause 5: The method according to any preceding clause, wherein the other data and video are temporally correlated with each other via timestamps linking segments of video to detected events that occurred during the medical procedure.
Clause 6: The method according to any preceding clause, further comprising generating by the AI analysis engine one or more of a recommendation for addressing a detected or predicted safety issue associated with the medical procedure, instructions for controlling the autonomous operations of the avatar or robotic system to facilitate the medical procedure, and a training dataset for further training artificial intelligence operations of the avatar or the robotic system.
Clause 7: The method according to any preceding clause, wherein the autonomous operations of the avatar that are controlled comprise one or more of assisting a user with inquiries, providing information to the user in response to the inquiries, auditorily presenting information output from the AI analysis engine, and responding to commands from the user.
Clause 8: The method according to any preceding clause, wherein the autonomous operations of the robotic system that are controlled comprise one or more of navigating a medical tool relative to an anatomy of a patient.
Clause 9: The method according to any preceding clause, wherein the AI analysis engine comprises a cascaded reasoning train architecture.
Clause 10: The method according to any preceding clause, further comprising further comprising dynamically changing an artificial intelligence model being used by the AI analysis engine during the medical procedure without altering the cascaded reasoning train architecture.
Clause 11: The method according to any preceding clause, wherein control of the autonomous operations of the avatar or the robotic system is further based on one or more of data received from a wearable device worn by a medical professional facilitating the medical procedure, data received from an invasive or non-invasive brain-interface device associated with the medical professional facilitating the medical procedure, and data received from a electro-mechanical control device actuated by the medical professional facilitating the medical procedure.
Clause 12: A system, comprising: an avatar or a robotic system; an artificial intelligence (AI) analysis engine; a processor configured to process and use video of a medical procedure in progress to control autonomous operations of the avatar or the robotic system; and a non-transitory computer-readable medium that stores instructions. The non-transitory computer-readable medium is configured to, when executed by the processor, cause the processor to perform operations comprising: obtaining a maximum number of input tokens that are allowed to be ingested by the AI analysis engine per session for a video quality associated with the video; determining a maximum desired temporal duration for a video segment based on the maximum number of input tokens and data analytics for other data to be input into an AI analysis engine along with video; determining a total number of video segments that the video can be partitioned into based on the video temporal duration and the maximum desired temporal duration; determining possible temporal durations for the video segments; and segmenting the video into a plurality of video segments based on the total number of video segments, the possible temporal durations for the video segments, and/or task types associated with frames of the video. The AI analysis engine analyzes a video segment of the plurality of video segments to monitor compliance of the medical procedure, automate documentation of the medical procedure, and/or detect or predict a safety issue during the medical procedure. The medical procedure is continued by controlling autonomous operations of the avatar or the robotic system based on at least an output of the AI analysis engine.
Clause 13: The system according to clause 12, wherein the processor is further caused to select a sampling rate for the video based on a type of medical procedure associated with the video.
Clause 14: The system according to any preceding system clause, wherein the processor is further caused to identify the AI analysis engine from a plurality of AI analysis engines trained to generate notes for a medical procedure of a type associated with the video.
Clause 15: The system according to any preceding system clause, wherein the processor is further caused to provide the video segment of the plurality of video segments to the AI analysis engine for use in generating the medical procedure notes.
Clause 16: The system according to any preceding system clause, wherein the processor is further caused to temporally correlate the other data and video with each other via timestamps linking segments of video to detected events that occurred during the medical procedure.
Clause 17: The system according to any preceding system clause, wherein the AI analysis engine generates one or more of a recommendation for addressing a detected or predicted safety issue associated with the medical procedure, instructions for controlling the autonomous operations of the avatar or robotic system to facilitate the medical procedure, and a training dataset for further training artificial intelligence operations of the avatar or the robotic system.
Clause 18: The system according to any preceding system clause, wherein the autonomous operations of the avatar that are controlled comprise one or more of assisting a user with inquiries, providing information to the user in response to the inquiries, auditorily presenting information output from the AI analysis engine, and responding to commands from the user.
Clause 19: The system according to any preceding system clause, wherein the autonomous operations of the robotic system that are controlled comprise one or more of navigating a medical tool relative to an anatomy of a patient.
Clause 20: The system according to any preceding system clause, wherein the AI analysis engine comprises a cascaded reasoning train architecture.
Clause 21: The system according to any preceding system clause, wherein the processor is further caused to dynamically change an artificial intelligence model being used by the AI analysis engine during the medical procedure without altering the cascaded reasoning train architecture.
Clause 22: The system according to any preceding system clause, wherein control of the autonomous operations of the avatar or the robotic system is further based on one or more of data received from a wearable device worn by a medical professional facilitating the medical procedure, data received from an invasive or non-invasive brain-interface device associated with the medical professional facilitating the medical procedure, and data received from a electro-mechanical control device actuated by the medical professional facilitating the medical procedure.

The terms “processor” and “processing device” refer to a hardware component of an electronic device that is configured to execute programming instructions. Except where specifically stated otherwise, the singular terms “processor” and “processing device” are intended to include both single-processing device embodiments and embodiments in which multiple processing devices together or collectively perform a process.

The terms “memory,” “memory device,” “computer-readable medium,” “data store,” “data storage facility” and the like each refer to a non-transitory device on which computer-readable data, programming instructions or both are stored. Except where specifically stated otherwise, the terms “memory,” “memory device,” “computer-readable medium,” “data store,” “data storage facility” and the like are intended to include single device embodiments, embodiments in which multiple memory devices together or collectively store a set of data or instructions, as well as individual sectors within such devices. A computer program product is a memory device with programming instructions stored on it.

As used in this document, the singular form “a”, “an”, and “the” include plural references unless the context clearly dictates otherwise. Unless defined otherwise, all technical and scientific terms used herein have the same meanings as commonly understood by one of ordinary skill in the art. As used in this document, the term “comprising” means “including, but not limited to”.

The described features, advantages and characteristics disclosed herein may be combined in any suitable manner. One skilled in the relevant art will recognize, in light of the description herein, that the disclosed systems and/or methods can be practiced without one or more of the specific features. In other instances, additional features and advantages may be recognized in certain scenarios that may not be present in all instances.

Although the systems and methods have been illustrated and described with respect to one or more implementations, equivalent alterations and modifications will occur to others skilled in the art upon the reading and understanding of this specification and the annexed drawings. In addition, while a particular feature may have been disclosed with respect to only one of several implementations, such feature may be combined with one or more other features of the other implementations as may be desired and advantageous for any given or particular application. Thus, the breadth and scope of the disclosure herein should not be limited by any of the above descriptions. Rather, the scope of the invention should be defined in accordance with the following claims and their equivalents.

What is claimed is:

1. A method for processing and using video of a medical procedure in progress, comprising:
obtaining, by a processor, a maximum number of input tokens that are allowed to be ingested by an artificial intelligence (AI) analysis engine per session for a video quality associated with the video;
determining, by the processor, a maximum desired temporal duration for a video segment based on the maximum number of input tokens and data analytics for other data to be input into the AI analysis engine along with video;
determining, by the processor, a total number of video segments that the video can be partitioned into based on the video temporal duration and the maximum desired temporal duration;
determining, by the processor, possible temporal durations for the video segments;
segmenting, by the processor, the video into a plurality of video segments based on the total number of video segments, the possible temporal durations for the video segments, and/or task types associated with frames of the video;
analyzing, by the AI analysis engine, a video segment of the plurality of video segments to monitor compliance of the medical procedure, automate documentation of the medical procedure, and/or detect or predict a safety issue during the medical procedure; and
continuing the medical procedure by controlling autonomous operations of an avatar or a robotic system based on at least an output of the AI analysis engine.

2. The method according to claim 1, further comprising selecting a sampling rate for the video based on a type of medical procedure associated with the video.

3. The method according to claim 1, further comprising identifying the AI analysis engine from a plurality of AI analysis engines trained to generate notes for a medical procedure of a type associated with the video.

4. The method according to claim 1, further comprising providing the video segment of the plurality of video segments to the AI analysis engine for use in generating the medical procedure notes.

5. The method according to claim 1, wherein the other data and video are temporally correlated with each other via timestamps linking segments of video to detected events that occurred during the medical procedure.

6. The method according to claim 1, further comprising generating by the AI analysis engine one or more of a recommendation for addressing a detected or predicted safety issue associated with the medical procedure, instructions for controlling the autonomous operations of the avatar or robotic system to facilitate the medical procedure, and a training dataset for further training artificial intelligence operations of the avatar or the robotic system.

7. The method according to claim 6, wherein the autonomous operations of the avatar that are controlled comprise one or more of assisting a user with inquiries, providing information to the user in response to the inquiries, auditorily presenting information output from the AI analysis engine, and responding to commands from the user.

8. The method according to claim 1, wherein the autonomous operations of the robotic system that are controlled comprise one or more of navigating a medical tool relative to an anatomy of a patient.

9. The method according to claim 1, wherein the AI analysis engine comprises a cascaded reasoning train architecture.

10. The method according to claim 9, further comprising further comprising dynamically changing an artificial intelligence model being used by the AI analysis engine during the medical procedure without altering the cascaded reasoning train architecture.

11. The method according to claim 1, wherein control of the autonomous operations of the avatar or the robotic system is further based on one or more of data received from a wearable device worn by a medical professional facilitating the medical procedure, data received from an invasive or non-invasive brain-interface device associated with the medical professional facilitating the medical procedure, and data received from a electro-mechanical control device actuated by the medical professional facilitating the medical procedure.

12. A system, comprising:
an avatar or a robotic system;
an artificial intelligence (AI) analysis engine;
a processor configured to process and use video of a medical procedure in progress to control autonomous operations of the avatar or the robotic system; and
a non-transitory computer-readable medium that stores instructions that is configured to, when executed by the processor, cause the processor to perform operations comprising:
obtaining a maximum number of input tokens that are allowed to be ingested by the AI analysis engine per session for a video quality associated with the video;
determining a maximum desired temporal duration for a video segment based on the maximum number of input tokens and data analytics for other data to be input into an AI analysis engine along with video;
determining a total number of video segments that the video can be partitioned into based on the video temporal duration and the maximum desired temporal duration;
determining possible temporal durations for the video segments; and
segmenting the video into a plurality of video segments based on the total number of video segments, the possible temporal durations for the video segments, and/or task types associated with frames of the video;
wherein the AI analysis engine analyzes a video segment of the plurality of video segments to monitor compliance of the medical procedure, automate documentation of the medical procedure, and/or detect or predict a safety issue during the medical procedure; and
wherein the medical procedure is continued by controlling autonomous operations of the avatar or the robotic system based on at least an output of the AI analysis engine.

13. The system according to claim 12, wherein the processor is further caused to select a sampling rate for the video based on a type of medical procedure associated with the video.

14. The system according to claim 12, wherein the processor is further caused to identify the AI analysis engine from a plurality of AI analysis engines trained to generate notes for a medical procedure of a type associated with the video.

15. The system according to claim 12, wherein the processor is further caused to provide the video segment of the plurality of video segments to the AI analysis engine for use in generating the medical procedure notes.

16. The system according to claim 12, wherein the processor is further caused to temporally correlate the other data and video with each other via timestamps linking segments of video to detected events that occurred during the medical procedure.

17. The system according to claim 12, wherein the AI analysis engine generates one or more of a recommendation for addressing a detected or predicted safety issue associated with the medical procedure, instructions for controlling the autonomous operations of the avatar or robotic system to facilitate the medical procedure, and a training dataset for further training artificial intelligence operations of the avatar or the robotic system.

18. The system according to claim 17, wherein the autonomous operations of the avatar that are controlled comprise one or more of assisting a user with inquiries, providing information to the user in response to the inquiries, auditorily presenting information output from the AI analysis engine, and responding to commands from the user.

19. The system according to claim 12, wherein the autonomous operations of the robotic system that are controlled comprise one or more of navigating a medical tool relative to an anatomy of a patient.

20. The system according to claim 12, wherein the AI analysis engine comprises a cascaded reasoning train architecture.

21. The system according to claim 20, wherein the processor is further caused to dynamically change an artificial intelligence model being used by the AI analysis engine during the medical procedure without altering the cascaded reasoning train architecture.

22. The system according to claim 12, wherein control of the autonomous operations of the avatar or the robotic system is further based on one or more of data received from a wearable device worn by a medical professional facilitating the medical procedure, data received from an invasive or non-invasive brain-interface device associated with the medical professional facilitating the medical procedure, and data received from a electro-mechanical control device actuated by the medical professional facilitating the medical procedure.
